(19) United States
(12) Patent Application Publication
(10) Pub. No.: US 2026/0294864 A1
(43) Pub. Date: Oct. 1, 2026

(54) PHARMACEUTICAL COMPOSITION FOR THE TREATMENT AND PREVENTION OF HPV

(71) Applicant: NP1 SRL, Roma (IT)

(72) Inventors: Roberto BECELLI, Roma (IT); Federico DE MARCO, Roma (IT); Fabio ALTIERI, Roma (IT)

(21) Appl. No.: 19/100,680

(22) PCT Filed: Aug. 3, 2023
(86) PCT No.: PCT/IT2023/050191

§ 371 Date: Feb. 3, 2025

(30) Foreign Application Priority Data
Aug. 4, 2022 (IT) 102022000016647

(51) Int. Cl. A61K 31/352; A61K 31/192; A61K 31/216; A61K 31/353; A61K 31/7048; A61P 31/20

(52) U.S. Cl. CPC A61K 31/352; A61K 31/192; A61K 31/216; A61K 31/353; A61K 31/7048; A61P 31/20

(57) ABSTRACT

The present invention concerns a pharmaceutical composition for the treatment and prevention of HPV infection and/or related cancer, wherein said composition is able to counteract the implantation of HPV transformed cells and their subsequent proliferation and tissue invasion.

The present invention concerns a pharmaceutical composition for the prevention and treatment of HPV infection and related lesions, wherein said composition is able to prevent the implantation of HPV transformed cells and suppress their subsequent proliferation and clonal expansion.

HPV infection is a common viral infection causing benign proliferative lesions (Condylomas or warts) of skin or mucous epithelia. There are more than 200 types whose infection is transmitted either by direct skin-to-skin or by direct mucosa-to-mucosa contact.

Most of HPV infections are completely clinically unapparent. In a part of cases however small proliferative lesions are generated that persist for months and are ultimately spontaneously cleared. Nevertheless, it can severely impact on patients' wellness depending on its localization. There are the following major clinical presentations:
I. Genital Condylomas. This is the most diffused Sexual Transmitted Disease (STD) worldwide. It is mostly caused by the HPV types 6 and 11 and consists in the appearance of flat lesions, small cauliflower-like bumps, or tiny stemlike protrusions. In women, genital warts appear mostly on the vulva but can also occur near the anus, on the cervix or in the vagina. In men, genital warts appear on the penis and scrotum or around the anus. Genital warts rarely cause discomfort or pain, though they may itch or feel tender. Persist for 6-18 months and then are spontaneously cleared. Nonetheless, they are generally source of deep anxiety, discomfort and shame and severely affect the personal and intimate life.
II. Common warts. Common warts, mostly caused by HPV-1, HPV-2, HPV-5 and others, appear as rough, raised cauliflower-like bumps and usually occur on the hands and fingers. In most cases, common warts are simply unsightly, but they can also be painful or susceptible to injury or bleeding.
III. Plantar warts. Plantar warts are usually caused by HPV types 1, -2, -3, -4, -27, -29, -57, with the HPV-1 assumed to be the most prevalent one. They are hard, grainy growths that usually appear on the heels or balls of your feet. These warts might cause discomfort.
IV. Flat warts. Flat warts are flat-topped, slightly raised lesions. They can appear anywhere, but children usually get them on the face and men tend to get them in the beard area. Women tend to get them on the legs.
V. Laryngeal Papillomatosis. Children born to mother infected by anogenital Condylomas (either clinically apparent or inapparent) may develop, in the early childhood, the Juvenile Larynx Condylomatosys. This is a biologically benign paediatric productive laryngeal infection mostly due to the HPV-11 and characterized by the growth of vocal cords condylomas. These lesions can be so large to compromise vocal expression, food assumption and cause progressive respiratory distress with the potential risk of suffocation. The surgical removal of laryngeal condylomas is mandatory but recurrency rate is so high (nearly 40%) that children undergoing multiple surgical treatments per year are not uncommon.
VI. Dysplastic cervico-vaginal lesions. HPV infection of the cervico-vaginal epithelia is a very common occurrence estimated to concern any woman at least once in life. Once these lesions are sustained by a few types of the AlphaPapillomavirus genus, the so-called High-Risk HPV (HR-HPV), moderate to severe dysplastic lesions are generated. These lesions tend to persist and may eventually give rise to invasive cervical cancer. Although cancer is indeed a rare complication of viral infection, yet the very high prevalence of infection makes it a rather common occurrence. Indeed, cervical cancer is the third, most common cause of neoplastic death for women at the global level, and the 8^{th} cause of neoplastic death in Italy and Southern Europe. In addition to the obvious burden of personal anxiety, pain, and deaths of any kind of neoplastic disease, cervical carcinoma, because of its peak of incidence in the 3^{rd}-5^{th} decades of life, affects middle aged women in the most productive and qualitative period of their lives thus posing a very high burden on society, families, and communities.
VII. Oropharyngeal cancers. It is now unanimously recognized that HR-HPV infection plays a driving role also in a subset of oropharyngeal cancers and in tonsillar cancers whose incidence is now dramatically increasing.
VIII. Anorectal dysplastic lesions and anorectal cancer. With mechanisms partly related to those occurring in cervico-vaginal lesions HR-HPVs are also the cause of anorectal cancer. This is a condition largely diffused and bitterly under-explored and under-diagnosed among middle-aged homosexual men and among male and female HIV patients.

HPV specific vaccines have been released at the beginning of the 21^{st} century. These vaccines have excellent safety and efficacy profiles holding the potentials for a complete eradication of any kind of both infectious and neoplastic HR-HPV related conditions. However, despite such a favorable context vaccinal campaigns are dramatically failing. Indeed, because of economic, social, and cultural reasons, coverage rates are largely unsatisfactory in any region, and figures are steadily worsening (Gabutti G et al 2021). Sadly, poor vaccine coverages mostly occur in medium and low-income countries and among those social groups and geographical areas where HPV infections and related cancers are more prevalent. Thus, for the next decades more than 50% of world population is expected to remain fully susceptible to a number of potentially preventable neoplastic disease.

A second line, non-vaccine-based tool for the prevention of HR-HPV related conditions is provided by the early diagnosis of pre-neoplastic lesions. Indeed, since the establishment of a HR-HPV infection a long and complex series of events is needed for the development of a full neoplastic phenotype. This process, known as the neoplastic progression, usually takes years to complete and is accompanied, at least in the case of cervical cancer, by a distinct pattern of progressively severe cyto-histological signs. Based on these signs the early detection of preneoplastic lesions can be easily accomplished largely before the beginning of invasive growth.

However, once a dysplastic cervical lesion has been detected, it must be surgically removed. A few different technical approaches are available to this end, mostly consisting in LASER excision, electrosurgical procedures (called Loop Electrosurgical Excision Procedure) (LEEP) or Cold Knife Conization. Each of them is rather conservative, minimally invasive, and adequate to day-surgery setting. Conversely all of them need to be implemented by highly qualified, and hence numerically limited, surgical equips, are expensive and technically demanding and, worse, all of them are burdened by a high recurrency rate so that a close follow-up is needed often mandating repeated surgery sessions. Such a high recurrency rate is due both to the uncomplete removal of the primary lesions and to their secondary implantation cells because of the sampling/removal procedures. These very same facts are also the reasons for the high recurrency rate of the Juvenile Laryngeal Papillomatosis mentioned above.

Sadly, the same sociological and psychological negative attitude toward HPV vaccination also apply to cervical screening campaigns that are actually strictly followed just by a minority of the general population. Moreover, it must be considered that the option of early detection of pre-neoplastic lesions is not available for the cases of anorectal, oro-pharyngeal, and laryngeal cancers because no specific cyto-histological marker has been so far identified for pre-neoplastic lesions in these districts.

Thus, the burden of HPV related condition is expected to persist for years at the global level. Thus, treatments able to reduce/counteract the implantation of HPV transformed cell, to restrict or limit the proliferation of already established lesions and to prevent their secondary spreading/invasion are highly needed.

According to the above considerations, new solutions for the prevention and treatment of HPV-related cancers are urgently needed. Such new solutions must be able to overcome the pitfalls of currently available methods.

It is known that polyphenols are a large family of molecules, largely diffused in food, nutraceuticals and in officinal plants. They are very popular and credited for a wide range of pharmacological properties including antioxidant, antimicrobial, antidiabetic, cell protective and antineoplastic activity. (Leri M et al 2020) (Sur S et al 2017) (Zhou Y et al 2016) (Cardona F et al 2013).

Flavonoids are a polyphenols sub-class of plant's secondary metabolic product extensively found in fruits and vegetables and reported to have a variety of immune-modulatory, anti-inflammatory, and antimicrobial functions in mammals (Kozkowska A et al 2014) (Kopustinskiene D M et al 2020).

The number of scientific reports dealing with potentially antineoplastic effects of Polyphenols and Flavonoids is steadily increasing (Sur S et al 2017) (Zhou Y et al 2016), however available data depict a rather heterogeneous and sometimes contradictory landscape and no univocal indication can be drawn. Additionally, a very few reports are presently available addressing the metastatic process or the related field of secondary implantation of transformed pre-neoplastic cells. During metastasis, cancer cells migrate from the primary site of clonal growth, invade the local extracellular matrix (ECM), enter the blood/lymph stream, migrate through the matrix of a topographically distant and histologically different tissues, and establish a secondary neoplastic growth. This requires a coordinated and sequential activation and deactivation of many specialized cell functions, including, but not limited to, adhesion, cytoskeleton remodeling, matrix digestion and deposition, and polarization/depolarization. Indeed, Gallic acid, a polyphenol prototype, was shown to reduce the migration of gastric cancer cells (Ho H H et al 2010) and glioma cells (Lu J et al 2010) in wound-healing and Boyden Chamber assays. Caffeic acid was found to reduce the invasiveness of PC3 prostate cancer cells by 50% (Lansky E P et al 2005). Abel et al (2018a; 2018b) reported that some purified polyphenolic components could partially reduce the cell adhesion in PC3 and DU145 human prostate cancer cells on collagen-I (but not on fibronectin-1) coated plastics and reduce the matrigel invasion by PC3 cells but not DU145 cells. However, all these effects were of limited extent and could be elicited only by very high concentrations invariably associated with severe toxic effects.

According to the present invention, it has now been surprisingly found that a few polyphenolic compounds are effective against HPV.

In particular, according to the present invention, 16 polyphenols/flavonoid compounds (hereafter named also RNP), listed in Table 1, were tested as potential components of a new pharmacological formulation for the treatment of HPV-related conditions.

TABLE 1

| | |
|---|---|
| 1 | Vanillic acid |
| 2 | Caffeic acid |
| 3 | Coumaric acid |
| 4 | Ferulic acid |
| 5 | Benzoic acid |
| 6 | Chlorogenic acid |
| 7 | Protocatechuic acid |
| 8 | Pinocembrin |
| 9 | Kaempferol |
| 10 | Galangin |
| 11 | Pinobanksin |
| 12 | Luteolin |
| 13 | Rutin |
| 14 | Chrysin |
| 15 | Quercetin |
| 16 | Apigenin |

Interestingly, among these 16 candidates, six were shown to have a distinct anti-clonal effect. Namely, Kaempferol (9), Galangin (10) and Luteolin (12) proved to have a sharp anticlonogenic and antiproliferative effect on HPV transformed cells. According to the present invention, these three molecules can provide a core composition for an innovative treatment to prevent the implantation of HPV transformed cells and their subsequent proliferation and tissue invasion. Specifically, the combination of Kaempferol (9), Galangin (10) and Luteolin (12) shows synergistic effects against HPV infection and related diseases, as described in Example 1.

In addition, according to the present invention, a second line group of molecules consisting in Chrysin (14), Quercetin (15) and Apigenin (16) have also shown fair anti-clonal effect, although milder than the one of the former three compounds, and may represent a complementary set of substances to improve the pharmacological profile of the core composition. Indeed, their association can elicit a distinct synergistic effect, i.e., their combination induces the same or a higher grade of inhibitory effect than the one induced by the isolated compounds. These data are recapitulated in Table 2.

TABLE 2

| | Molecule | CID₅₀ | | IRR |
|---|---|---|---|---|
| Major Active | Kaempferol | 25 | μM | 2 |
| polyphenols | Galangin | 25 | μM | 2 |
| | Luyeolin | 6.25 | μM | 8 |
| Minor Active | Chrysin | 25 | μM | 2 |
| polyphenols | Quercetin | 50 | μM | 1 |
| | Apigenin | 25 | μM | 2 |
| Synergistic | Kaempferol/Apigenin | 12.5/12.5 | μM | 2 |
| associations | Chrysin/Quercetin | 25/3.125 | μM | 2 |
| | Luteolin/Galangin | 6.25/25 | μM | 2 |
| | Luteolin/Chrysin | 6.25/25 | μM | 2 |
| | Luteolin/Quercetin | 6.25/25 | μM | 2 |
| | Luteolin/Apigenin | 6.25/25 | μM | 2 |
| | Kaempferol/galangin/ | 12.5/12.5/6.25 | μM | 2 |
| | Luteolin | | | |

Table 2 shows the anti-clonal effect of isolated polyphenols and their associations. The inhibitory relative rate (IRR) (fourth column) provides an efficacy ranking of molecules. Assuming the Quercetin, i.e., the less active molecule, as the efficacy unit, the IRR is calculated as the reverse ratio of the CID₅₀ of a probing molecule to the reference CID₅₀ of Quercetin.

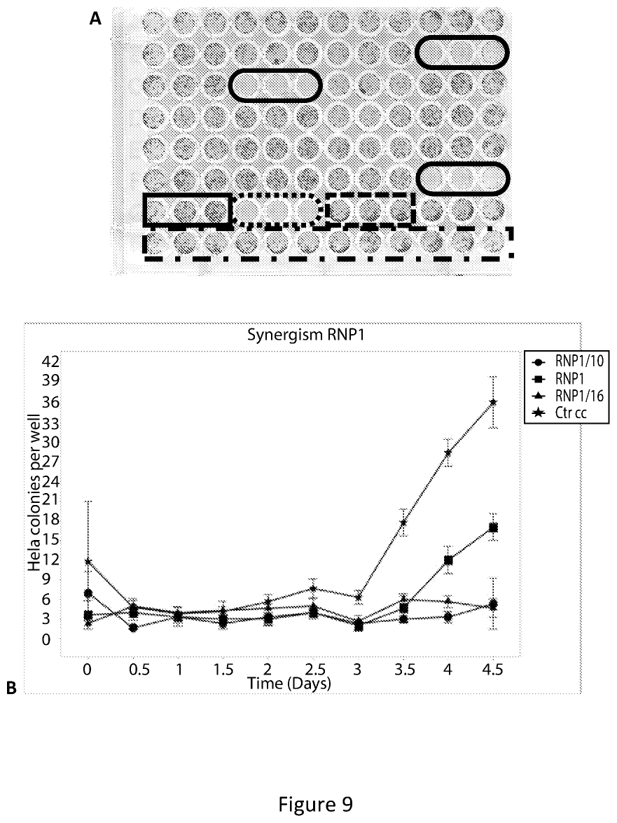

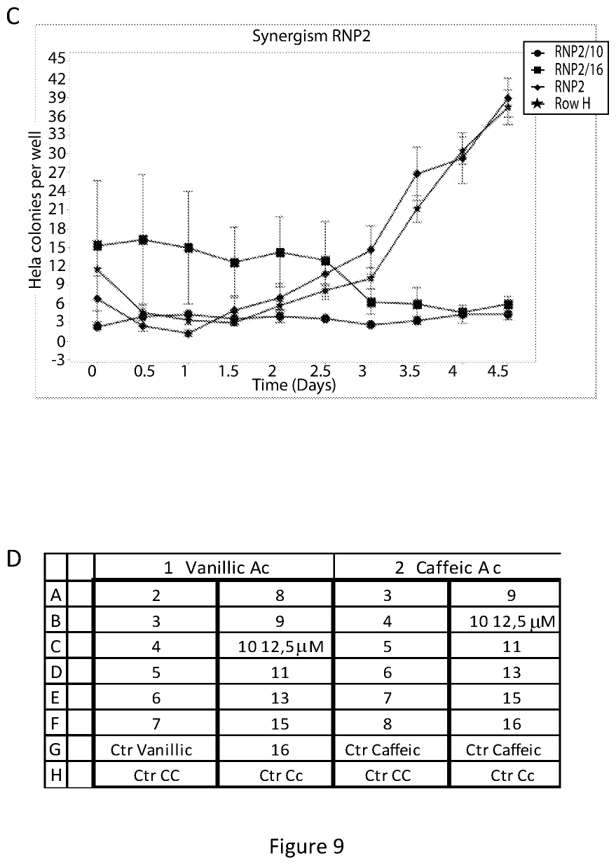

Finally, according to the present invention, it has also been found that the other molecules i.e., Vanillic acid (1) and Caffeic acid (2), although per se devoid of any direct activity, once combined with Galangin or Quercetin, are able to induce a certain grade of clonal inhibition and the same is also true for Coumaric (3), Ferulic (4), Benzoic (5), Chlorogenic (6) and Protocatechuic (7) acids, once combined with Galangin, Chrysin or Apigenin (FIG. 9-11). This third set of compounds, may be of potential interest as potentiating agents.

Therefore, the present invention advantageously provides a pharmaceutical composition of low molecular weight polyphenol/flavonoid compounds able to prevent the implantation, the clonal expansion and the secondary migration of HPV transformed neoplastic cells. The composition according to the invention is aimed to prevent the initiation of HPV transformation; to provide a topical medical treatment for established HPV related lesions; to prevent their recurrence following surgical excision.

More specifically, the pharmaceutical composition according to the present invention can be advantageously used for:
the prevention of secondary implantation of HPV lesions following to Colposcopy and surgical removal of CINII/CINIII cervical lesions.
the prevention of secondary implantation of HPV lesions following other endoscopic/diagnostic or surgical removal of dysplastic/neoplastic lesions in the oropharyngeal and laryngeal cavity.
the prevention of primary implantation and of secondary spreading of HPV lesions following odonto-stomatological procedures in patients with a history of dysplastic/neoplastic lesions of the Oropharyngeal and laryngeal cavity.
the prevention of HR-HPV infection of the female genital tract following unshielded/unprotected sexual intercourses.
the unpainful, self-administered medical treatment of CIN-I or unapparent HR-HPV infection of the female genital tract. A rather common condition for which only the “watch and wait option” is currently available.
the medical treatment of the Juvenile Laryngeal Recurrent Papillomatosis, a biologically benign potentially fatal condition.
the prevention of anal HPV infection and the following dysplastic/neoplastic lesions in HIV positive patients and among homosexual males, a condition difficult to suspect and to diagnose and presently in increasingly faster rise.
The prevention of iatrogenic transmission of HR-HPV infection during endoscopic diagnostic procedures
The prevention of Genital condylomas recurrence following their surgical excision.

It is therefore specific object of the present invention a pharmaceutical composition comprising or consisting of luteolin, galangin and kaempferol, together with one or more pharmaceutically acceptable excipients and/or adjuvants.

Luteolin, galangin and kaempferol compounds according to the present invention can be either synthetic compounds or extracted from natural sources, such as plants.

In the pharmaceutical composition according to the present invention,
luteolin can be present in an amount ranging from 1 to 20 μM, for example from 1 to 10 μM, preferably from 2 to 12 μM, for example from 2 to 8 μM, more preferably from 4 to 7 μM,
galangin can be present in an amount ranging from 10 to 40 μM, preferably from 20 to 40 μM, more preferably from 20 to 30 μM, even more preferably 25 μM,
kaempferol can be present in an amount ranging from 10 to 40 μM, preferably from 20 to 40 μM, such as from 20 to 30 μM, more preferably 22 to 30 μM, even more preferably 12 to 30 μM, for example 25 μM or 15 μM.

According to the present invention, said pharmaceutical composition can further comprise one, more than one or all the compounds selected from chrysin, quercetin and apigenin.

Chrysin, quercetin and apigenin compounds according to the present invention can be synthetic compounds or they can be isolated from natural sources, such as plants.

In the pharmaceutical composition according to the present invention, chrysin can be present in an amount ranging from 10 to 50 μM, preferably from 15 to 30 μM, more preferably 20 μM or 25 μM, quercetin can be present in an amount ranging from 40 to 80 μM, preferably from 50 to 60 μM, more preferably 50 μM, apigenin can be present in an amount ranging from 10 to 50 μM, preferably from 10 to 40 μM, more preferably from 15 to 30 μM, more preferably 25 μM.

According to an embodiment of the present invention, said pharmaceutical composition can further comprise one, more than one or all compounds selected from vanillic acid, caffeic acid, coumaric acid, ferulic acid, benzoic acid, chlorogenic acid, protocatechuic acid, pinocembrin, rutin and pinobanksin, preferably one, more than one or all compounds selected from vanillic acid, caffeic acid, coumaric acid, ferulic acid, benzoic acid, chlorogenic acid, protocatechuic acid, more preferably caffeic acid and/or ferulic acid.

Vanillic acid, caffeic acid, coumaric acid, ferulic acid, benzoic acid, chlorogenic acid, protocatechuic acid, pinocembrin, rutin and pinobanksin compounds according to the present invention can be synthetic compounds or they can be isolated from plants or from other natural sources.

According to an embodiment of the present invention, said pharmaceutical composition is not honey.

According to an embodiment of the present invention, the pharmaceutical composition does not comprise other flavonoids or polyphenols different from luteolin, galangin and kaempferol.

According to a further embodiment of the present invention, said pharmaceutical composition does not comprise other flavonoids or polyphenols different from luteolin, galangin, kaempferol, chrysin, quercetin and apigenin.

According to a further embodiment of the present invention, said pharmaceutical composition does not comprise other flavonoids or polyphenols different from luteolin, galangin, kaempferol, chrysin, quercetin, apigenin, vanillic acid, caffeic acid, coumaric acid, ferulic acid, benzoic acid, chlorogenic acid, protocatechuic acid.

According to a further embodiment of the present invention, said pharmaceutical composition does not comprise other flavonoids or polyphenols different from luteolin, galangin, kaempferol, chrysin, quercetin, apigenin, vanillic acid, caffeic acid, coumaric acid, ferulic acid, benzoic acid, chlorogenic acid, protocatechuic acid, pinocembrin, rutin and pinobanksin.

According to the present invention, each of vanillic acid, coumaric acid, benzoic acid, chlorogenic acid, protocatechuic acid, pinocembrin, rutin and pinobanksin compounds can be present in an amount <0,100 mM, for example <0,050, whereas each of Caffeic acid and Ferulic Acid compounds is present in an amount from 20 to 100 μM.

The present invention also concerns the pharmaceutical composition as defined above for medical use.

In addition, the present invention concerns the pharmaceutical composition as defined above for use in the prevention and treatment of a viral infection and/or related disease.

According to the present invention, the viral infection can be HPV infection, such as human Alpha HPV infection, and the related disease can be chosen from the group consisting of HPV-related cancer, HPV dysplastic lesions, HPV laryngeal papillomatosis, HPV Genital Condylomata, AlphaPapillomavirus sub-clinical anogenital and cervico-vaginal infection and AlphaPapillomavirus latent anogenital and cervico-vaginal infection (i.e.: the mere molecular positivity).

According to an embodiment of the present invention, said pharmaceutical composition, for the above-mentioned uses, is not honey.

According to the present invention, said pharmaceutical composition can be administered by a route chosen from the group consisting of topical route, intravenous route, intramuscular route, organ selective extracorporeal perfusion, intracavital perfusion, intra peritoneal perfusion, intracerebral ventricles or sub-arachnoid continuous perfusion.

In addition, according to the present invention, said pharmaceutical composition can be delivered by liposomes, nanoparticles; radiofrequencies, electroporation, ionophoresis or shockwaves delivery systems.

It is a further object of the present invention a combination of luteolin, galangin and kaempferol for separate and sequential use in the prevention and treatment of a viral infection and/or related disease.

The combination according to the present invention, for the above-mentioned separate or sequential use, can further comprise one, more than one or all the compounds selected from chrysin, quercetin and apigenin.

In addition, the combination according to the present invention, for the above-mentioned separate or sequential use, can further comprise one, more than one or all compounds selected from vanillic acid, caffeic acid, coumaric acid, ferulic acid, benzoic acid, chlorogenic acid, protocatechuic acid, pinocembrin rutin and pinobanksin; preferably one, more than one or all compounds selected from vanillic acid, caffeic acid, coumaric acid, ferulic acid, benzoic acid, chlorogenic acid, protocatechuic acid, more preferably caffeic acid and/or ferulic acid.

According to the present invention, “separate use” is understood as meaning the administration, at the same time, of the compounds of the combination according to the invention, wherein each compound is in a distinct pharmaceutical form.

According to the present invention, “sequential use” is understood as meaning the successive administration of the compounds of the combination according to the invention, wherein each compound is in a distinct pharmaceutical form.

The separate and sequential uses are intended in the order of hours, such as in the same day, the same morning, the same afternoon. In other words, all the compounds of the combination have to be administered as part of a single medical prescription, in a period of time so that their therapeutical effects can combine to each other and achieve their synergistic effect.

According to the combination of the invention, the viral infection can be HPV infection, such as human Alpha HPV infection, and the related disease can be chosen from the group consisting of HPV-related cancer, HPV dysplastic lesions, HPV laryngeal papillomatosis, HPV Genital condylomata, AlphaPapillomavirus sub-clinical anogenital and cervico-vaginal infection and AlphaPapillomavirus unapparent anogenital and cervico-vaginal infection (i.e.: the mere molecular positivity) According to the present invention, the compounds of the combination can be administered by a route chosen from the group consisting of topical route, intravenous route, intramuscular route, organ selective extracorporeal perfusion, intracavital perfusion, intra peritoneal perfusion, intracerebral ventricles or sub-arachnoid continuous perfusion.

According to the present invention, the compounds of the combination can be delivered by liposomes, nanoparticles; radiofrequencies, electroporation, ionophoresis or shockwaves delivery systems.

The present invention also concerns one or more compounds chosen from luteolin, galangin, kaempferol, chrysin, quercetin and apigenin for use in the prevention and treatment of a viral infection and/or related disease.

According to the invention, luteolin, galangin, kaempferol, chrysin, quercetin and apigenin can be synthetic compounds or they can be isolated from other natural sources, such as plants.

According to the present invention, said viral infection can be HPV infection, such as human Alpha HPV infection, and said related disease can be chosen from the group consisting of HPV-related cancer, HPV dysplastic lesions, HPV laryngeal papillomatosis, HPV Genital condylomata, AlphaPapillomavirus sub-clinical anogenital and cervico-vaginal infection and AlphaPapillomavirus unapparent anogenital and cervico-vaginal infection (i.e.: the mere molecular positivity).

According to the present invention, said more compounds can be chosen from the group consisting of kaempferol and galangin; kaempferol and luteolin; kaempferol and chrysin; kaempferol and quercetin; kaempferol and apigenin; galangin and luteolin; galangin and chrysin; galangin and quercetin; galangin and apigenin; luteolin and chrysin; luteolin and quercetin; luteolin and apigenin; chrysin and quercetin; chrysin and apigenin; quercetin and apigenin; kaempferol, galangin and luteolin; kaempferol, galangin and chrysin; kaempferol, galangin and quercetin; kaempferol, galangin and apigenin; kaempferol, luteolin and chrysin; kaempferol, luteolin and quercetin; kaempferol, luteolin and apigenin; kaempferol, chrysin and quercetin; kaempferol, chrysin and apigenin; kaempferol, quercetin and apigenin; galangin, luteolin and chrysin; galangin, luteolin and quercetin; galangin, luteolin and apigenin; galangin, chrysin and quercetin; galangin, chrysin and apigenin; galangin, quercetin and apigenin; luteolin, chrysin and quercetin; luteolin, chrysin and apigenin; luteolin, quercetin and apigenin; chrysin, quercetin and apigenin; kaempferol, galangin, luteolin and chrysin; kaempferol, galangin, luteolin and quercetin; kaempferol, galangin, luteolin and apigenin; kaempferol, galangin, chrysin and quercetin; kaempferol, galangin, chrysin and apigenin; kaempferol, galangin, quercetin and apigenin; kaempferol, luteolin, chrysin and quercetin; kaempferol, luteolin, chrysin and apigenin; kaempferol, luteolin, quercetin and apigenin; kaempferol, chrysin, quercetin and apigenin; galangin, luteolin, chrysin and quercetin; galangin, luteolin, chrysin and apigenin; galangin, luteolin, quercetin and apigenin; galangin, chrysin, quercetin and apigenin; luteolin, chrysin, quercetin and apigenin; kaempferol, galangin, luteolin, chrysin and quercetin; kaempferol, galangin, luteolin, chrysin and apigenin; kaempferol, galangin, luteolin, quercetin and apigenin; kaempferol, galangin, chrysin, quercetin and apigenin; kaempferol, luteolin, chrysin, quercetin and apigenin; galangin, luteolin, chrysin, quercetin and apigenin; kaempferol, galangin, luteolin, chrysin, quercetin and apigenin; preferably kaempferol, galangin and luteolin; chrysin and quercetin; kaempferol and apigenin; galangin and luteolin; chrysin and luteolin; or luteolin and apigenin.

The present invention also concerns a pharmaceutical composition comprising one or more compounds chosen from luteolin, galangin, kaempferol, chrysin, quercetin and apigenin, together with one or more pharmaceutically acceptable excipients and/or adjuvants, for use in the prevention and treatment of a viral infection and/or related disease. According to the present invention, the viral infection can be HPV, such as human Alpha HPV infection, and the related disease can be chosen from the group consisting of HPV-related cancer, HPV dysplastic lesions, HPV laryngeal papillomatosis, HPV Genital condylomata, AlphaPapillomavirus sub-clinical anogenital and cervico-vaginal infection and AlphaPapillomavirus unapparent anogenital and cervico-vaginal infection (i.e.: the mere molecular positivity).

According to an embodiment of the present invention, said pharmaceutical composition, for the above-mentioned uses, is not honey.

According to the present invention, said more compounds in the pharmaceutical composition can be chosen from the group consisting of kaempferol and galangin; kaempferol and luteolin; kaempferol and chrysin; kaempferol and quercetin; kaempferol and apigenin; galangin and luteolin; galangin and chrysin; galangin and quercetin; galangin and apigenin; luteolin and chrysin; luteolin and quercetin; luteolin and apigenin; chrysin and quercetin; chrysin and apigenin; quercetin and apigenin; kaempferol, galangin and luteolin; kaempferol, galangin and chrysin; kaempferol, galangin and quercetin; kaempferol, galangin and apigenin; kaempferol, luteolin and chrysin; kaempferol, luteolin and quercetin; kaempferol, luteolin and apigenin; kaempferol, chrysin and quercetin; kaempferol, chrysin and apigenin; kaempferol, quercetin and apigenin; galangin, luteolin and chrysin; galangin, luteolin and quercetin; galangin, luteolin and apigenin; galangin, chrysin and quercetin; galangin, chrysin and apigenin; galangin, quercetin and apigenin; luteolin, chrysin and quercetin; luteolin, chrysin and apigenin; luteolin, quercetin and apigenin; chrysin, quercetin and apigenin; kaempferol, galangin, luteolin and chrysin; kaempferol, galangin, luteolin and quercetin; kaempferol, galangin, luteolin and apigenin; kaempferol, galangin, chrysin and quercetin; kaempferol, galangin, chrysin and apigenin; kaempferol, galangin, quercetin and apigenin; kaempferol, luteolin, chrysin and quercetin; kaempferol, luteolin, chrysin and apigenin; kaempferol, luteolin, quercetin and apigenin; kaempferol, chrysin, quercetin and apigenin; galangin, luteolin, chrysin and quercetin; galangin, luteolin, chrysin and apigenin; galangin, luteolin, quercetin and apigenin; galangin, chrysin, quercetin and apigenin; luteolin, chrysin, quercetin and apigenin; kaempferol, galangin, luteolin, chrysin and quercetin; kaempferol, galangin, luteolin, chrysin and apigenin; kaempferol, galangin, luteolin, quercetin and apigenin; kaempferol, galangin, chrysin, quercetin and apigenin; kaempferol, luteolin, chrysin, quercetin and apigenin; galangin, luteolin, chrysin, quercetin and apigenin; kaempferol, galangin, luteolin, chrysin, quercetin and apigenin; preferably kaempferol, galangin and luteolin; chrysin and quercetin; kaempferol and apigenin; galangin and luteolin; chrysin and luteolin; or luteolin and apigenin

In the pharmaceutical composition according to the invention as defines above
luteolin can be present in an amount ranging from 1 to 20 μM, for example from 1 to 10 μM, preferably from 2 to 12 μM, for example from 2 to 8 μM, more preferably from 4 to 7 μM,
galangin can be present in an amount ranging from 10 to 40 μM, preferably from 20 to 40 μM, more preferably from 20 to 30 μM, even more preferably 25 μM,
kaempferol can be present in an amount ranging from 10 to 40 μM, preferably from 20 to 40 μM, such as from 20 to 30 μM, more preferably 22 to 30 μM, even more preferably 12 to 30 μM, for example 25 μM or 15 μM,
chrysin can be present in an amount ranging from 10 to 50 μM, preferably from 15 to 30 μM, more preferably 20 μM or 25 μM.
quercetin can be present in an amount ranging from 40 to 80 μM, preferably from 50 to 630 μM, more preferably 50 μM.
apigenin can be present in an amount ranging from 10 to 50 μM, preferably from 10 to 40 μM, more preferably from 15 to 30 μM, more preferably 25 μM.

The pharmaceutical composition according to the invention can further comprise one, more than one or all compounds selected from vanillic acid, caffeic acid, coumaric acid, ferulic acid, benzoic acid, chlorogenic acid, protocatechuic acid, pinocembrin, rutin and pinobanksin; preferably caffeic acid, ferulic acid, vanillic acid, coumaric acid, benzoic acid, chlorogenic acid, protocatechuic acid, more preferably Caffeic acid and/or Ferulic acid.

For example, said pharmaceutical composition can comprise vanillic acid, galangin and apigenin; caffeic acid, galangin and apigenin; coumaric acid, galangin, chrysin and apigenin; ferulic acid, galangin, chrysin and apigenin; benzoic acid, galangin, chrysin and apigenin; chlorogenic acid, galangin, chrysin and apigenin; or protocatechuic acid, galangin, chrysin and apigenin.

As mentioned above, vanillic acid, caffeic acid, coumaric acid, ferulic acid, benzoic acid, chlorogenic acid, protocatechuic acid, pinocembrin, rutin and pinobanksin compounds according to the present invention can be synthetic compounds or they can be isolated from natural sources, such as plants.

According to the present invention, each of vanillic acid, coumaric acid, benzoic acid, chlorogenic acid, protocatechuic acid, pinocembrin, rutin and pinobanksin compounds can be present in an amount <0,100 mM, for example <0,050 mM, whereas each of Caffeic acid and Ferulic acid compounds is present in an amount from 20 to 100 μM.

According to the present invention, said pharmaceutical composition can be administered by a route chosen from the group consisting of topical route, intravenous route, intramuscular route, organ selective extracorporeal perfusion, intracavital perfusion, intra peritoneal perfusion, intracerebral ventricles or sub-arachnoid continuous perfusion.

According to the present invention, said pharmaceutical composition can be delivered by liposomes, nanoparticles; radiofrequencies, electroporation, ionophoresis or shockwaves delivery systems.

The present invention also concerns a combination of one or more compounds chosen from luteolin, galangin, kaempferol, chrysin, quercetin and apigenin for separate or sequential use in the prevention and treatment of a viral infection and/or related disease. According to the invention, the viral infection can be HPV, such as human Alpha HPV infection, and the related disease can be chosen from the group consisting of HPV-related cancer, HPV dysplastic lesions, HPV laryngeal papillomatosis, HPV Genital Condylomata, AlphaPapillomavirus sub-clinical anogenital and cervico-vaginal infection and AlphaPapillomavirus unapparent anogenital and cervico-vaginal infection (i.e.: the mere molecular positivity).

The combination according to the present invention, for the above-mentioned separate or sequential use, can comprise one, more than one or all the compounds selected from luteolin, galangin, kaempferol, chrysin, quercetin and apigenin.

In addition, the combination according to the present invention, for the above-mentioned separate or sequential use, can further comprise one, more than one or all compounds selected from vanillic acid, caffeic acid, coumaric acid, ferulic acid, benzoic acid, chlorogenic acid, protocatechuic acid, pinocembrin, rutin and pinobanksin, preferably caffeic acid, ferulic acid, vanillic acid, coumaric acid, benzoic acid, chlorogenic acid, protocatechuic acid, more preferably Caffeic acid and/or Ferulic acid.

According to the present invention, “separate use” is understood as meaning the administration, at the same time, of the compounds of the combination according to the invention, wherein each compound is in a distinct pharmaceutical form.

According to the present invention, “sequential use” is understood as meaning the successive administration of the compounds of the combination according to the invention, wherein each compound is in a distinct pharmaceutical form.

In particular, the combination according to the present invention can comprise one or more compounds chosen in the group consisting of kaempferol and galangin; kaempferol and luteolin; kaempferol and chrysin; kaempferol and quercetin; kaempferol and apigenin; galangin and luteolin; galangin and chrysin; galangin and quercetin; galangin and apigenin; luteolin and chrysin; luteolin and quercetin; luteolin and apigenin; chrysin and quercetin; chrysin and apigenin; quercetin and apigenin; kaempferol, galangin and luteolin; kaempferol, galangin and chrysin; kaempferol, galangin and quercetin; kaempferol, galangin and apigenin; kaempferol, luteolin and chrysin; kaempferol, luteolin and quercetin; kaempferol, luteolin and apigenin; kaempferol, chrysin and quercetin; kaempferol, chrysin and apigenin; kaempferol, quercetin and apigenin; galangin, luteolin and chrysin; galangin, luteolin and quercetin; galangin, luteolin and apigenin; galangin, chrysin and quercetin; galangin, chrysin and apigenin; galangin, quercetin and apigenin; luteolin, chrysin and quercetin; luteolin, chrysin and apigenin; luteolin, quercetin and apigenin; chrysin, quercetin and apigenin; kaempferol, galangin, luteolin and chrysin; kaempferol, galangin, luteolin and quercetin; kaempferol, galangin, luteolin and apigenin; kaempferol, galangin, chrysin and quercetin; kaempferol, galangin, chrysin and apigenin; kaempferol, galangin, quercetin and apigenin; kaempferol, luteolin, chrysin and quercetin; kaempferol, luteolin, chrysin and apigenin; kaempferol, luteolin, quercetin and apigenin; kaempferol, chrysin, quercetin and apigenin; galangin, luteolin, chrysin and quercetin; galangin, luteolin, chrysin and apigenin; galangin, luteolin, quercetin and apigenin; galangin, chrysin, quercetin and apigenin; luteolin, chrysin, quercetin and apigenin; kaempferol, galangin, luteolin, chrysin and quercetin; kaempferol, galangin, luteolin, chrysin and apigenin; kaempferol, galangin, luteolin, quercetin and apigenin; kaempferol, galangin, chrysin, quercetin and apigenin; kaempferol, luteolin, chrysin, quercetin and apigenin; galangin, luteolin, chrysin, quercetin and apigenin; kaempferol, galangin, luteolin, chrysin, quercetin and apigenin; preferably kaempferol, galangin and luteolin; chrysin and quercetin; kaempferol and apigenin.

According to the present invention, the compounds of the combination can be administered by a route chosen from the group consisting of topical route, intravenous route, intramuscular route, organ selective extracorporeal perfusion, intracavital perfusion, intra peritoneal perfusion, intracerebral ventricles or sub-arachnoid continuous perfusion.

According to the present invention, the compounds of the combination can be delivered by liposomes, nanoparticles; radiofrequencies, electroporation, ionophoresis or shockwaves delivery systems.

The present invention now will be described by an illustrative, but not limitative way, according to preferred embodiments thereof, with particular reference to the examples and the enclosed drawings, wherein:

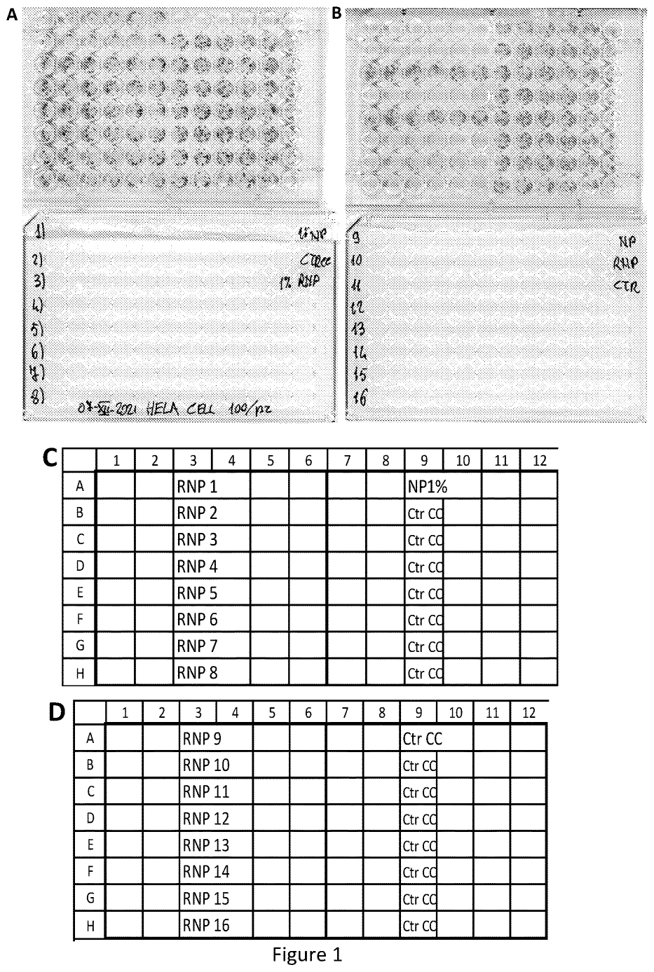

FIG. 1: Anti-clonal effect of RNP1-16 on HeLa cells.
Panel A): 50 μM of RNPs 1-8 were administered to cells in rows A-H, columns 1-6(six replicas per condition). None of compounds induced any noticeable effect as compared with control untreated cells (rows B-H, columns 7-12). An experimental positive control (NP) is shown in row A, wells 7-12.
Panel B): Anti-clonal effect of RNP 9-16. An almost complete suppression of clonal activity is clear with Kaempferol (9), Galangin (10) Luteolin (12), Chrysin (14), Quercetin (15) and Apigenin (16), respectively in raws A, B, D, F, G and H, columns 1-6. No effect can be seen with RNP11 Pinobanksin and RNP13 Rutin. Panel C and D: diagrammatic topographical maps of treatments and growth conditions on plates A and B respectively. Comparable results were obtained both on SiHa and ME-180 cells.

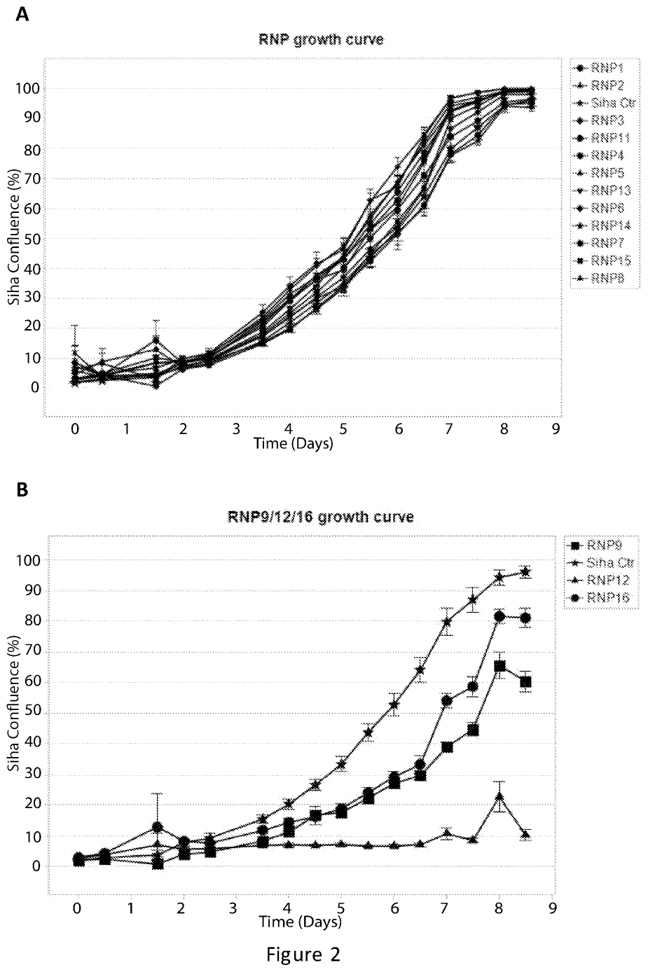

FIG. 2: live cell analysis of RNPs antiproliferative effect. Siha cells were seeded at 1000 cells/well in a 96 well plate and allowed to adhere overnight. The plate was then washed twice with PBS and replenished with 100 microliter/well fresh medium plus 50 μM of each compound. Images were taken every 12 hours; Growth curves were drawn plotting the percentage of confluence versus time. Panel A: No antiproliferative effect can be detected for RNP1-8,11 and 13-15. In fact, their growth curves outline a single spindle completely overlapping and masking the one of control untreated cells. Conversely, a distinct anti-proliferative effect is evident in panel B- where the growth curves of RNP 9 Kaempferol (solid squares), RNP 12 Luteolin (solid diamonds), RNP 16 Apigenin (solid circles) and control untreated cells (solid stars) are depicted. Luteolin induces an almost complete suppression of proliferation while a partial suppression, roughly of 20% and 40%, can be achieved by Apigenin and Kaempferol respectively. Each point represents the mean of six independent replicas. Bar width represents the Standard Deviation (SD). Consistent results were also obtained by HeLa and ME-180 cells

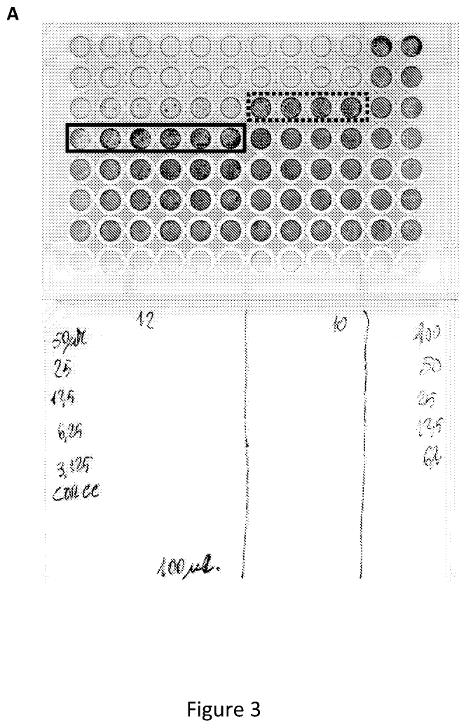

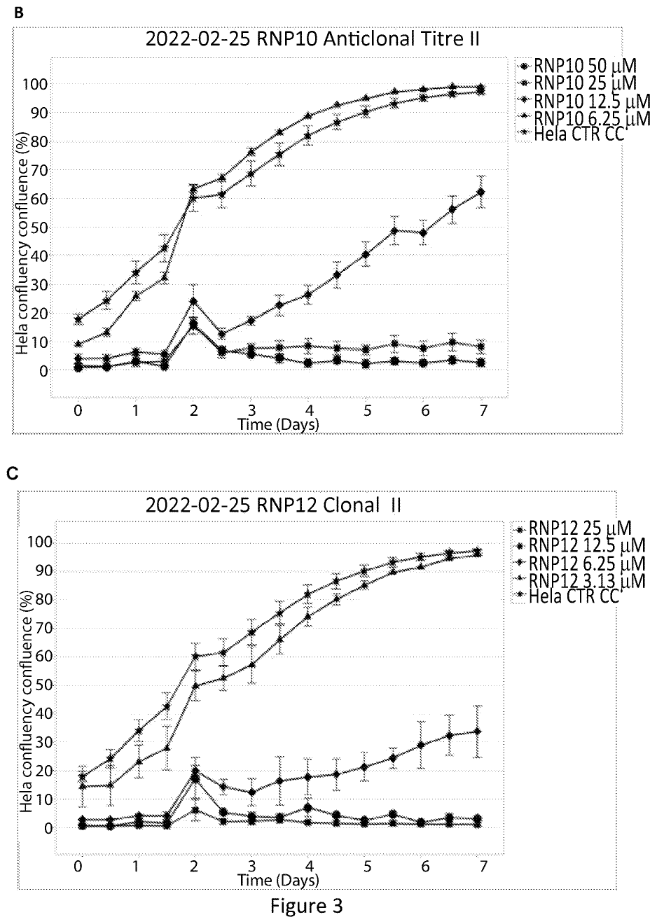

FIG. 3: Titration of Galangin and Luteolin anti-clonal effect. Panel A. Hela cells were challenged with serial twofold dilutions of 50 μM Luteolin (in columns 1-6) and 100 μM Galangin (columns 7-10). Hela control untreated cells in columns 11-12. A reduction of clonal activity is evident for Luteolin (solid box) and Galangin (dot box). The percentage of clonal inhibition is best evaluated through the percentage of confluence evaluated by the live cell image analysis shown in panels B and C that reveal a 6.25 μM CID₅₀ for Luteolin and of 12.5 μM for Galangin (solid and dotted boxes in panel A, respectively).

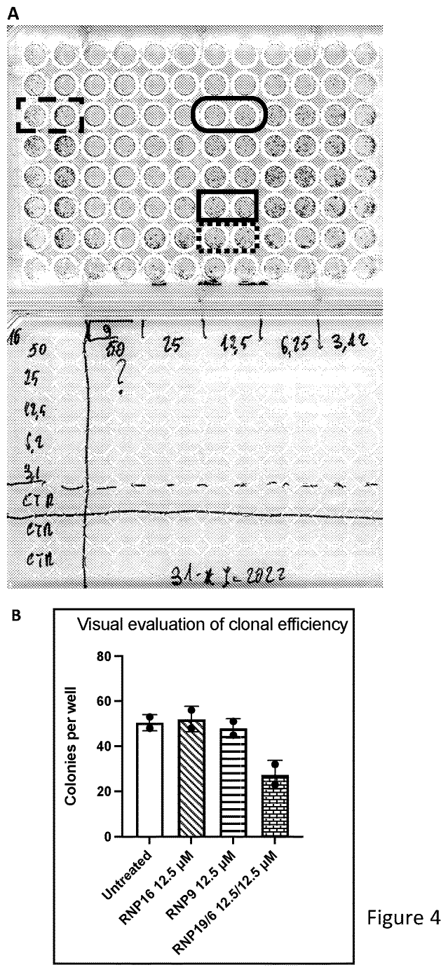

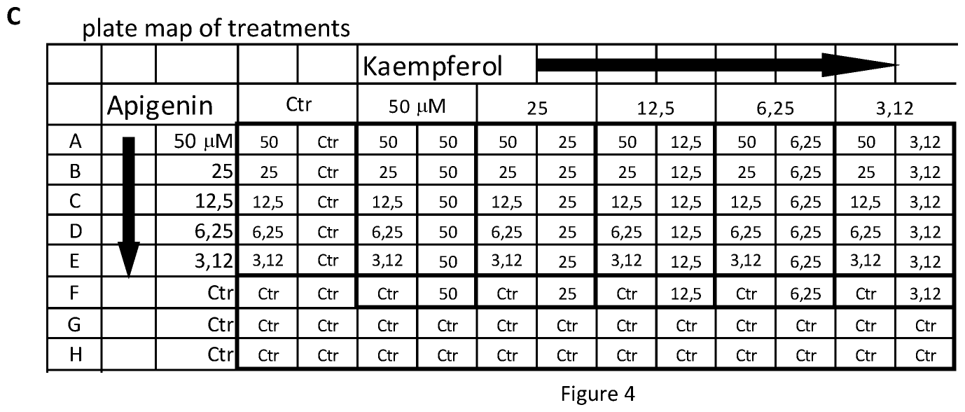

FIG. 4: Kaempferol/Apigenin synergistic activity. In this experiment Kaempferol and Apigenin were administered to Siha cells at decremental concentrations, either in combination or alone, as shown in the topographical map (in panel C). Cells treated with 12,5 μM Kaempferol (wells F: 6,7, solid box in panel A) or with 12.5 μM Apigenin (wells A:1,2 segmented box) showed no difference to control untreated cells (wells G: 6,7 dotted box). Interestingly their simultaneous administration at 12.5 μM each, induced a sharp reduction of the clonal efficiency (wells C: 6,7 solid oval). Histograms in Panel B depict the number of colonies per well as evaluated by visual inspection and count. Similar results were also obtained by HeLa and ME-180 cells

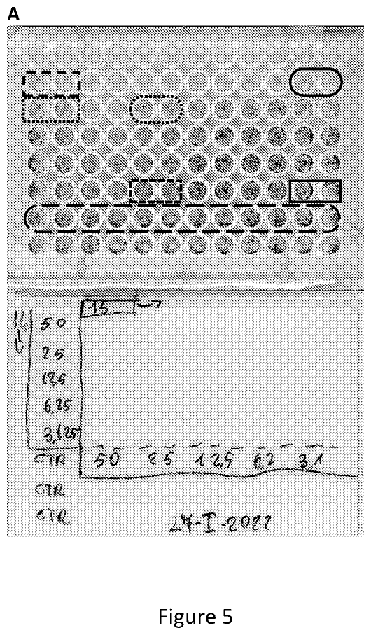

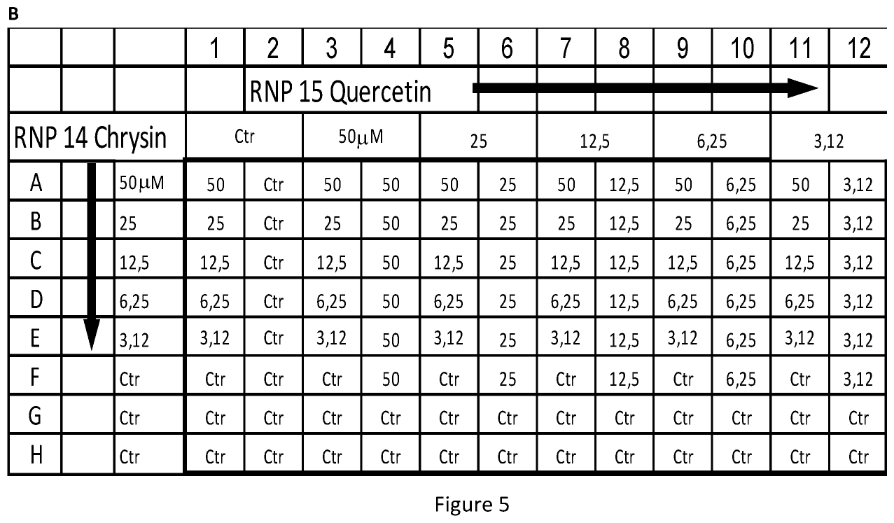

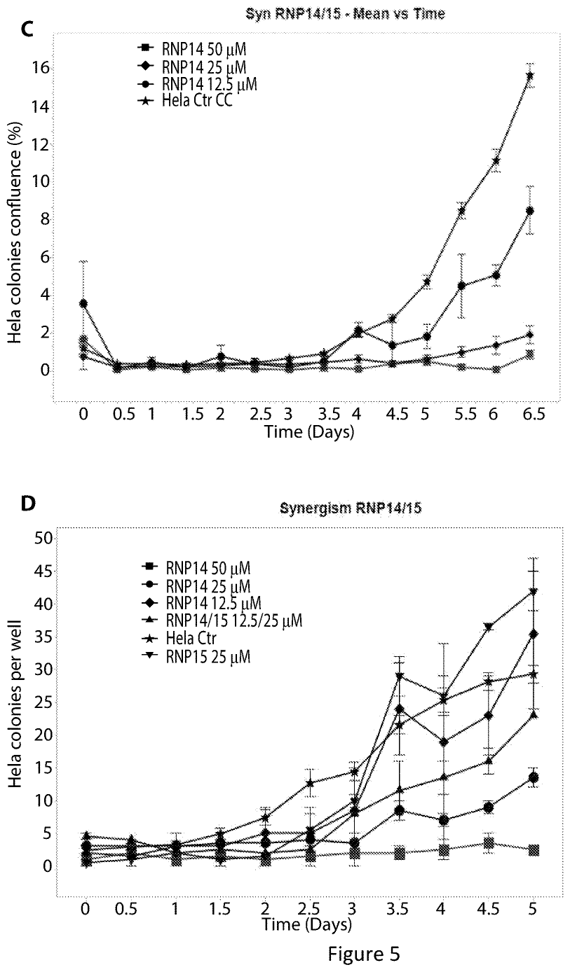

FIG. 5: Synergistic effect combined administration of Chrysin and Quercetin on HeLa cells. Panel A: end-point anti-clonal effect of Chrysin/Quercetin at 12,5/25 μM induce a clonal inhibition (dotted oval) stronger than those of isolated Chrysin (dotted Box) or quercetin (segmented box). Panel B: a diagrammatic topographical map of treatments and growth conditions on plates. Panel C and D Live cell analysis of conditions highlighted shown in panel A. The growth curve of Chrysin/Quercetin combination at 12,5/25 μM (triangle line in panel D) lays consistently below the curves of isolated Chrysin and Quercetin (circle line in panel C and inverted triangle line in panel D respectively). Almost superimposable results were also obtained in Ca-Ski cells

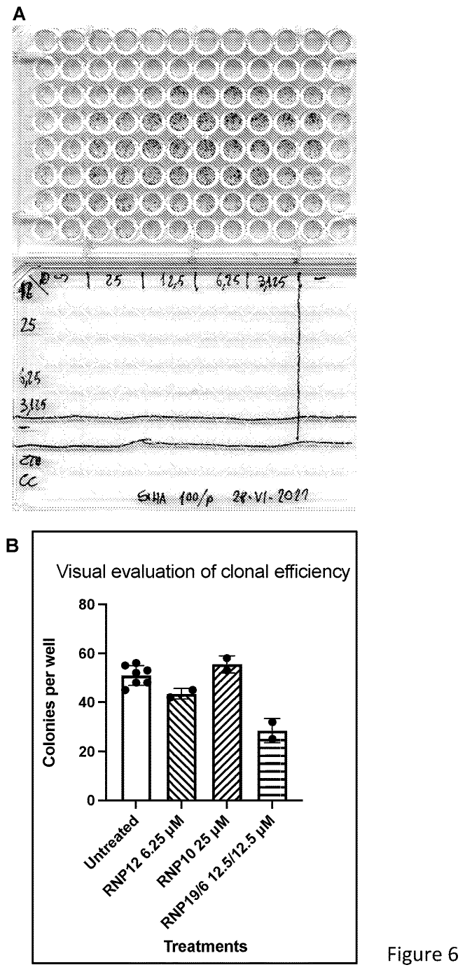

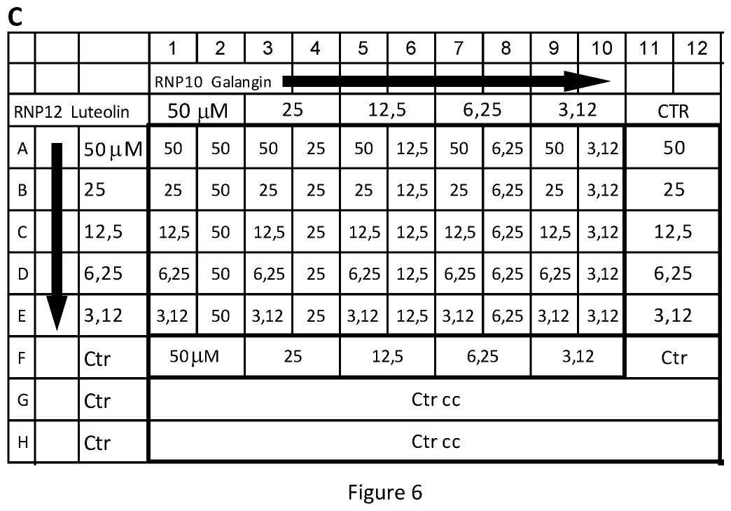

FIG. 6: Synergistic effect of the Galangin/Luteolin association. Panel A: end-point anti-clonal effect, on SiHa cells, of Galangin and Luteolin administered at decremental concentrations. Cells treated with 6.25 μM Luteolin (wells D:11,12, solid box in panel a) or with 25.0 μM Galangin (wells F:3,4, segmented box) showed no difference to control untreated cells (wells G: 3,9 segmented oval). Interestingly their simultaneous administration at 6.25/25.0 μM respectively, induced a clear reduction of the clonal efficiency (wells D:3,4, solid oval). The Histograms in Panel B depict the number of colonies per well as evaluated by visual inspection and count.
In Panel C a topographical map of plate treatments. Each condition was assayed in duplicate. Similar results were also obtained on HeLa cells.

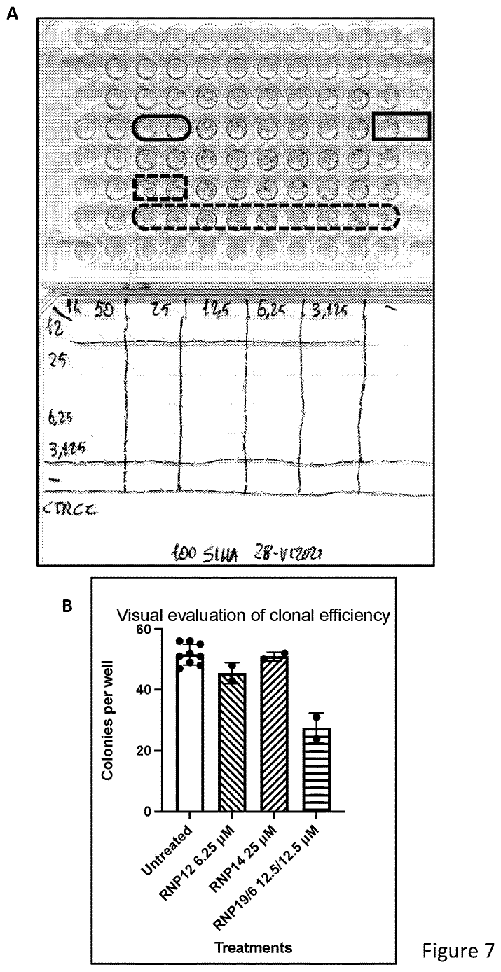

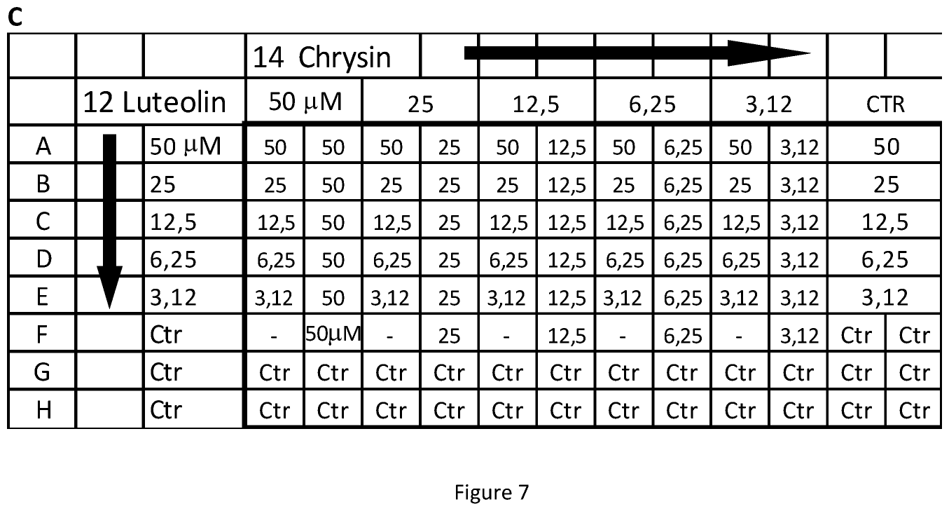

FIG. 7: Synergistic effect of the Chrysin/Luteolin. Panel A: end-point anticlonal effect on Siha cells of Chrysin and Luteolin administered at decremental concentrations. Cells treated with 6.25 μM Luteolin (wells D:11,12, solid box in panel a) or with 25.0 μM Galangin (wells F:3,4, segmented box) showed no difference to control untreated cells (wells G: 3,11 segmented oval). Interestingly their simultaneous administration at 6.25/25.0 μM respectively, induced a clear reduction of the clonal efficiency (wells D:3,4, solid oval). The number of colonies per well as evaluated by visual inspection and count is depicted by Histograms in Panel B. In Panel C a topographical map of plate treatments. Each condition was assayed in duplicate.

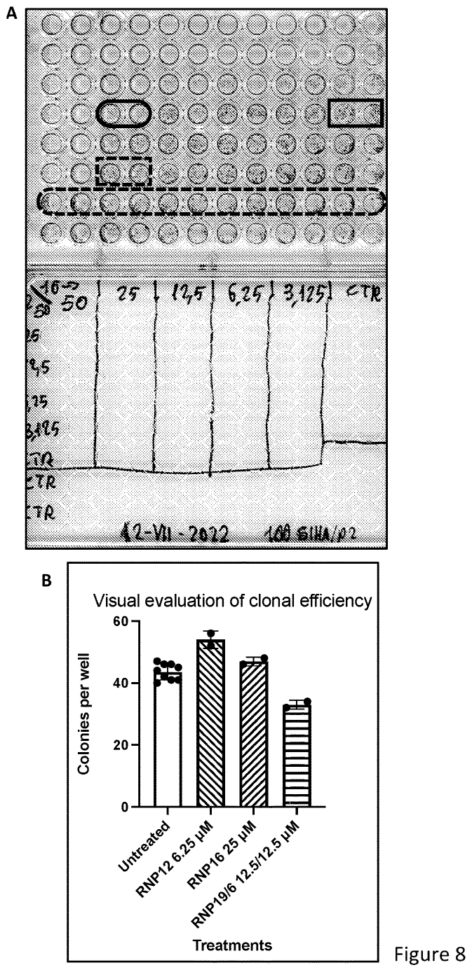

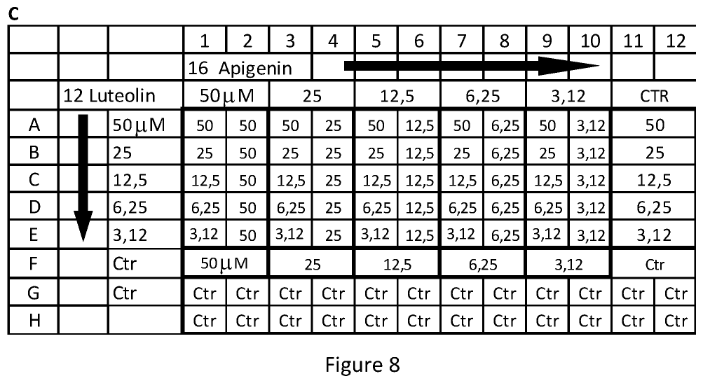

FIG. 8: Synergistic effect of the Luteolin/Apigenin association. Panel A: end-point anti-clonal effect of Luteolin and Apigenin administered to Siha cells at decremental concentrations. Cells treated with 6.25 μM Luteolin (wells D:11,12, solid box in panel a) or with 25.0 μM Apigenin (wells F:3,4, segmented box) showed no difference to control untreated cells (wells G: 1,12 segmented oval). Interestingly their simultaneous administration at 6.25/25.0 μM respectively, induced a clear reduction of the clonal efficiency (wells D:3,4, solid oval). In panel B Histograms depict the number of colonies per well as evaluated by visual inspection and count. In Panel C a diagrammatic topographical map of plate treatments. Each condition was assayed in duplicate.

FIG. 9: it shows that both Vanillic acid and Caffeic acid have a synergistic effect once administered together with Galangin and Apigenin. Hela cells were treated with either with Vanillic or Caffeic acid at 50 μM (left- and right-hand side of the plate respectively) in association with other compounds (25 μM each apart from Galangin that was administered at 12.5 μM). Panel A shows the end point clonal assay. In panel b a topographical sketch of treatments is shown. As it can be seen both Vanillic and Caffeic acids induced an almost complete clonal inhibition once combined with Galangin (solid ovals in panel A) or with Apigenin (dotted ovals in panel A) while neither of them induced any effect once compared with control untreated cells. Vanillic acid in solid box, Caffeic acid in segmented box, control untreated cells in the Line-dot-line box. For isolated effect of Galangin 12,5 μM and Apigenin 25.0 μM see FIG. 3, wells D:7-10 and FIG. 8, wells F:3,4, respectively. The extent of clonal inhibition is best evaluated through the percentage of confluence evaluated by the live cell image analysis shown in panels B and C. In Panel D a diagrammatic topographical map of plate treatments. Each condition was assayed in triplicate.

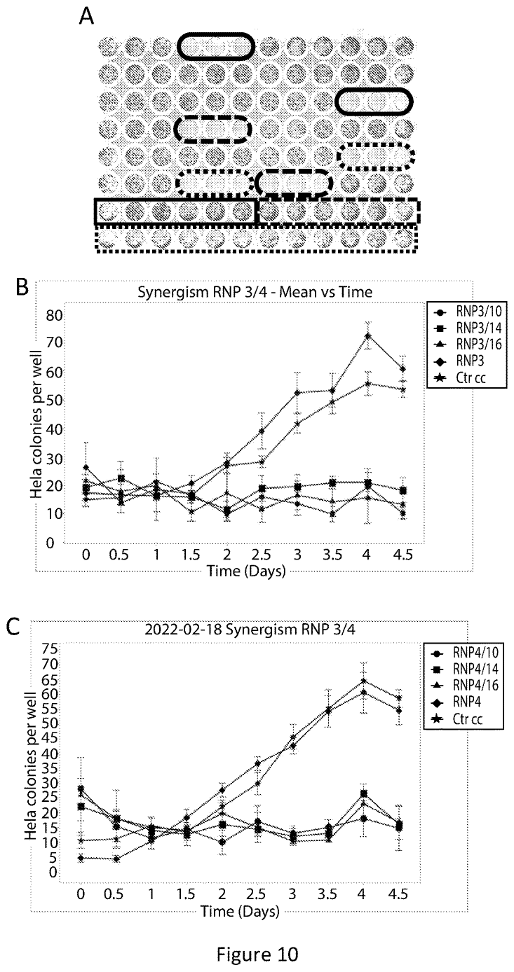

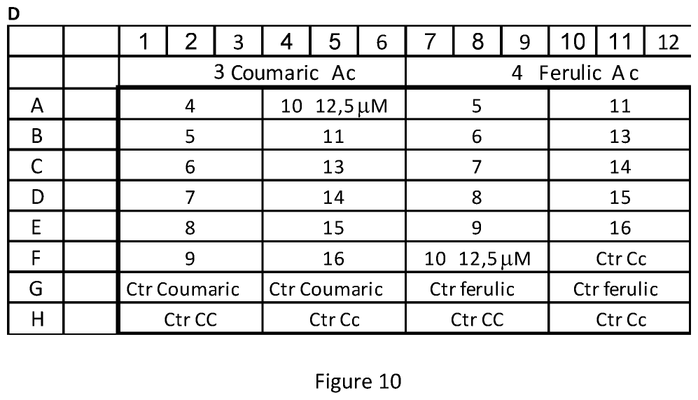

FIG. 10: Coumaric and Ferulic acids have a synergistic effect with Galangin, Chrysin and Apigenin. Hela cells were treated with Coumaric or Ferulic acids, at 50 μM (left- and right-hand side of the plate respectively), with other compounds at 25 μM each apart from Galangin that was administered at 12.5 μM. Panel A shows the end point clonal assay. As it can be seen both Coumaric acid and Ferulic acid induced an almost complete clonal inhibition of HeLa cells once combined with either Galangin (solid ovals) or Chrysin (segmented ovals), or Apigenin (dotted ovals). Neither of them induced any effect once administered alone: Coumaric acid in solid box, Ferulic acid in segmented box, control untreated cells in dotted box. For isolated effect of Galangin 12,5 μM, Chrysin 25.0 μM and Apigenin 25.0 μM see FIG. 3, wells D:7-10, FIG. 7, wells F:3,4 and FIG. 8, wells F:3,4 respectively. The extent of clonal inhibition is best evaluated through the percentage of confluence evaluated by the live cell image analysis shown in panels B and C. In Panel D a diagrammatic topographical map of plate treatments. Each condition was assayed in triplicate. Superimposable results were also obtained either with Ca-Ski or ME-180 cells.

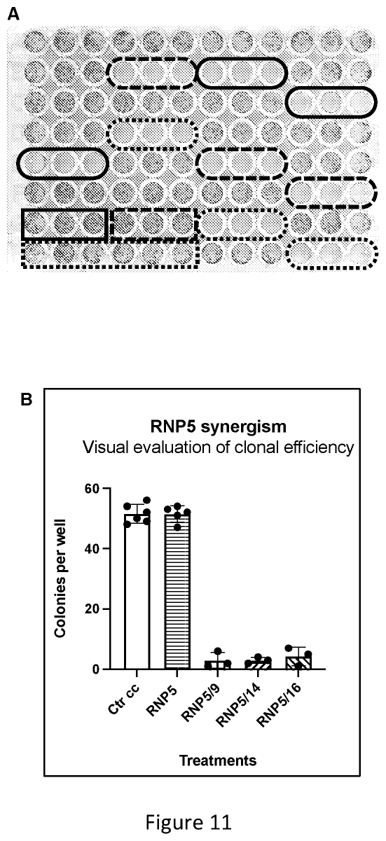

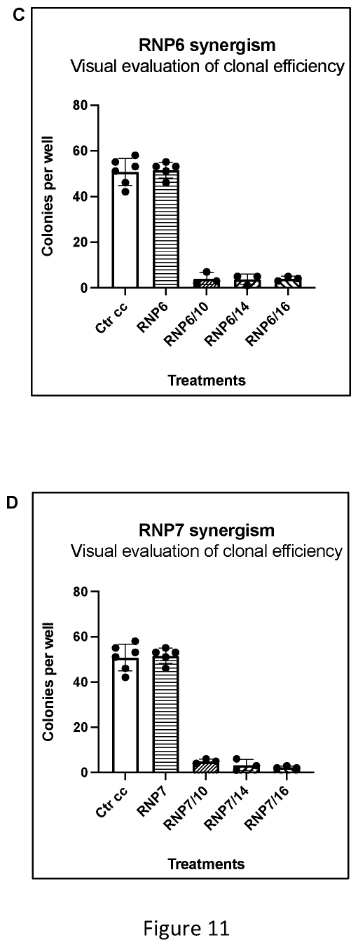

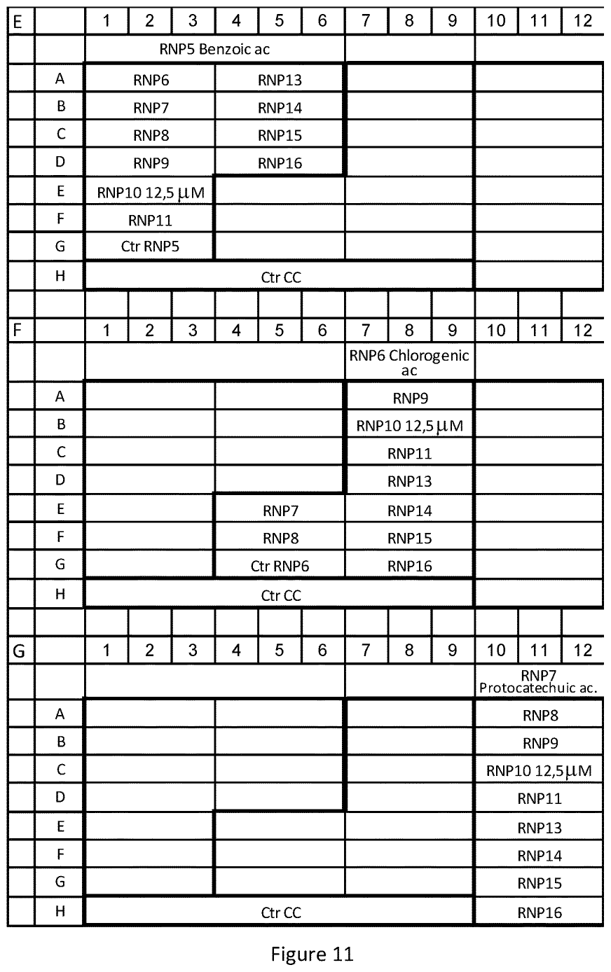

FIG. 11: Synergistic effect of Benzoic, Chlorogenic and Protocatechuic acids with Galangin, Chrysin and Apigenin. Hela cells were treated with Benzoic or Chlorogenic or protocatechuic acid, at 50 μM with other compounds at 25 μM each as sketched in panel E, F and G. As it can be seen their association with either Galangin (solid ovals) or Chrysin (segmented ovals), or Apigenin (dotted ovals) at 25 μM induced an almost complete clonal inhibition of HeLa cells. Conversely neither of them induced any effect once administered alone: Benzoic acid in solid box, Chlorogenic acid in segmented box, control untreated cells in dotted box. For isolated effect of Galangin 12,5 μM, Chrysin 25.0 μM and Apigenin 25.0 μM see FIG. 3, wells D:7-10, FIG. 7, wells F:3,4 and FIG. 8, wells F:3,4 respectively. Histograms in Panels B, C and D depict the number of colonies per well in highlighted conditions, as evaluated by visual inspection and count. Similar effects were also obtained using SiHa cells.

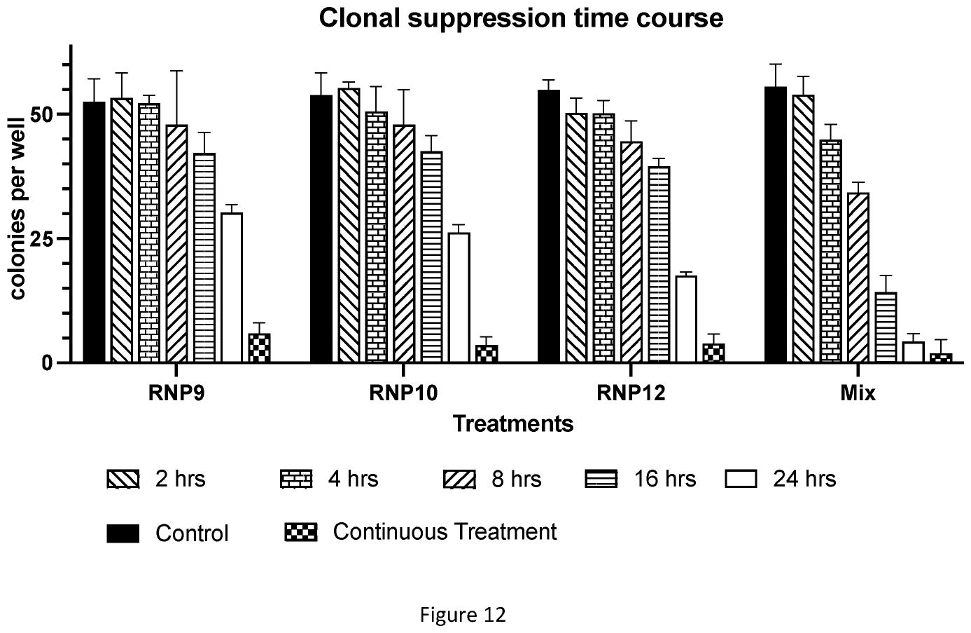

FIG. 12: Synergistic effect of simultaneous administration of Kaempferol, Galangin and Luteolin to Siha cells. Kaempferol, Galangin, Luteolin and their combination were administered to SiHa cells at the concentrations of 25 μM each and of 12,5/12,5/6,25 μM for their combination. Treatments were allowed to stay on cells either for 2 or 4/8/16/24 hours. After that, treatments were washed out, cells washed two times with PBS, replenished with fresh medium and the anti-clonal effect was then monitored by live cell image analysis.

As it can be seen almost 24 hours are needed to RNP9, RNP10 or RNP12 to induce a roughly 50% of clonal inhibition. Conversely, with the triple combination the clonal activity is readily inhibited with only a 8 hours treatment and its extent clearly exceed 50% with a 16 hours long treatment. Finally, the clonal inhibition achieved by the triple combination after 24 hours was much sharper than those of single components. The triple combination thus outperforms its single components in terms of a shorter time of induction and in terms of extent of effect. Similar results were also obtained with HeLa cells.

### EXAMPLE 1: STUDY OF THE EFFECTS AGAINST HPV OF POLYPHENOLIC COMPOUNDS ACCORDING TO THE PRESENT INVENTION

### Materials and Methods

The following molecules (hereafter also named RNP) have been assayed for antineoplastic activity.
1. Vanillic acid,
2. Caffeic acid
3. Coumaric acid
4. Ferulic acid
5. Benzoic acid
6. Chlorogenic acid
7. Protocatechuic acid
8. Pinocembrin
9. Kaempferol
10. Galangin
11. Pinobanksin
12. Luteolin
13. Rutin
14. Chrysin
15. Quercetin
16. Apigenin

### Chemicals

Polyphenols were obtained as 99% purified materials from Sigma-Aldrich Co (Merk Life Science Srl. Via Monte Rosa, 93. Milano—Italia).

### Cells

A total of 6 cell lines, outlined in Table 3, were used for this study. The cell lines were obtained from Research Centres of cell biology in Italy, United Kingdom and Germany. This research raised no ethical concern being based on entirely in vitro experiments using no patients' derived information or material. In compliance with National and International ethical rules, no informed consent is needed.

TABLE 3

| | | Origin and phenotypic | Cellosaurus | |
|---|---|---|---|---|
| Cell line | Host | description | accession no. | Ref |
| HeLa | Human | Epitheliod cells derived | CVCL_0030 | Scherer W F |
| | | from an invasive cervical | | et al 1953 |
| | | carcinoma harbouring | | |
| | | multiple copies of HPV18 | | |
| Siha | Human | Epithelioid cells from an | CVCL_0032 | Friedel F |
| | | invasive squamous | | et al 1970 |
| | | cervical carcinoma | | |
| | | harbouring a single, | | |
| | | incomplete HPV16 genome | | |
| Ca-Ski | Human | Epithelioid cells from | CVCL_1100 | Baker C C |
| | | invasive squamous | | et al 1987 |
| | | cervical carcinoma | | |
| | | hosting 500-800 copies | | |
| | | of largely rearranged | | |
| | | HPV16 genome. | | |
| HK-168 | Human | Human diploid | NA | De Marco F |
| | | keratinocytes in vitro | | et al 2007 |
| | | transformed with the full- | | |
| | | length HPV-16 genome | | |
| HaCaT | Human | Spontaneously | CVCL_0038 | Boukamp P |
| | | immortalised human | | et al 1988 |
| | | keratinocyte from a solar | | |
| | | dysplastic skin lesion of | | |
| | | the trunk of an adult male | | |
| | | patient | | |
| ME-180 | Human | Epithelioid cells from | CVCL_1401 | Yee C |
| | | cervical dysplastic lesion | | et al 1985. |
| | | hosting a few copies of | | |
| | | HPV68 genome. | | |

Legend: Cell line characteristics are fully described at www.cellosaurus.org. Accession numbers, where available, are reported in column 5. NA: not available.

HeLa cells are the oldest continuous cell lines and were originally derived from a HPV18 positive metastatic cervical carcinoma (Scherer W F et al 1953). Siha cells are an epithelioid like cell line derived from a HPV16-positive invasive cervical squamous carcinoma (Friedel F et al 1970). Ca-Ski cells are a line harboring multiple copy of transcriptionally active HPV16 genomes (Baker C C et al 1984). Both Hela and Siha were a kind gift of prof MS Campo, The Beatson Institute for Cancer Research, Glasgow, Scotland. These two cell lines because of their versatility and ease of use were used for most of experiments reported. Ca-Ski cells were obtained through the courtesy of prof. Matthias Durst, at the Deutsch Krebsforschungzentrum—Heidelberg, FRG. These cells used together with Siha provide a model for the genetic instability and variability of cervical cancers. HK-168 is an immortalized, non-tumorigenic, continuous cell line derived from human skin keratinocytes in vitro transformed with whole HPV16 genome (De Marco F et al 2007). This cell line is reputed to recapitulate le biological features of transformed dysplastic cells and was used in preliminary experiments to set the experimental dosages and conditions. The ME-180 cells are a cell line harboring a few, transcriptionally active, copies of HPV68 and provide an in vitro model for dysplastic/neoplastic lesions induced by HPV with Intermediate oncogenic Risk (Yee C C et al 1985). The HaCaT cells, derived from a solar skin lesion by Petra Boukamp et al (1988) are here used as an in vitro equivalent of Ultra-Violet induced pre-neoplastic epithelial cells to rule out possible direct toxic effects of polyphenols. Both HaCaT and ME-180, were kindly donated by Prof. Matthias Durst. Hela, Siha, Ca-Ski, ME-180 and HaCaT were all grown in high glucose DMEM supplemented with 10% fetal calf serum (FCS). The HK-168 cells, which retained the ability to terminally differentiate under high Ca²⁺ and FCS concentrations, were grown in K-SFM, a chemically defined medium supplemented with human EGF and bovine pituitary extract (ThermoFisher Scientific, Life Technologies, Monza—MI). Each cell line was sub-cultured twice a week at the appropriate split ratio according to the specific proliferative index. In no case antibiotics or antimycotics were added to the culture media. For the present study, all cell lines were retrieved from an in-house cell archive facility.

### Anti-Clonogenic Activity

The anti-clonogenic activity was evaluated both by the current standard qualitative plating efficiency assay and by quantitative live cell imaging analysis (INCUCYTE S3 Live Cell Analysis Imaging System—Sartorius Welwyn Garden City, Hertfordshire, UK). For qualitative plating efficiency assay cells were seeded at ‘clonal’ density (i.e., 0.5 cell/mm²) either in medium containing the testing molecules or with plain medium and incubated without any further manipulation. After 9-14 days, according to the cell line specific proliferative index, the cultures were decanted and stained with 0.2% CV/methanol for 5 min at room temperature. All conditions were tested in triplicates in a 6-well culture plate. The anti-clonogenic activity of the treatments was evaluated using the colony forming units (CFU) of treated versus control cultures. The minimal concentration which could reduce 50% of the CFU of treated versus control culture was defined as colony-forming Inhibitory concentration (CID₅₀). For quantitative evaluation cells were seeded in 96-well microplates at the above density and condition and allowed to grow the subsequent 8-day incubation in the INCUCYTE S3 Imaging System. Images were acquired at 12-hours intervals and colony formation and percentage of growth area covered (confluency) were then monitored through automated image analysis. The analysis parameters were as follows: segmentation adjustment=0.4; hole fill <5,000 μm²; adjusted pixel=−2. Objects were scored as colonies once made of at least 24 cells, i.e.: had an area above 24,000 μm² and an eccentricity of <0.750.

For antiproliferative effect cells were seeded in 96-well microplates at 1000 cells/per well (i.e.: 30 cell/mm²), a density allowing exponential growth rate for the subsequent 8-day incubation. After overnight adhesion cell monolayers were washed and replenished with fresh media with different testing molecules, plain medium served as cell growth control.

Cell growth was then monitored by automated INCUCTE S3 imaging Systems. Images were acquired at 12-hours intervals. The analysis parameters were as follows: segmentation adjustment=0.9; hole fill <7,000 μm²; adjusted pixel=0.

### Statistical Analysis

Data for growth curves, anti-clonal and Antiproliferative assays were obtained from at least three independent experiments. Each data point of quantitative anti-proliferative or anti-clonal experiments represents the mean (±SD) of eight independent replicates. Data were analyzed and plotted using Prism 6.0 software (GraphPad Software, San Diego, CA).

### Results

The anti-clonal effects of polyphenols on Hela cells are shown in FIG. 1. As it can be seen (panel a) the components 1) Vanillic acid, 2) Caffeic acid, 3) Coumaric acid, 4) Ferulic acid, 5) Benzoic acid, 6) Chlorogenic acid, 7) Protocatechuic acid and 8) Pinocembrin, used isolated, where devoid of any significant anticlonogenic effect. Conversely (in panel b) Kaempferol (9), Galangin (10) and Luteolin (12), Chrysin (14), Quercetin (15) and Apigenin (16), once administered at 50 μM, a concentration much lower than the one of polyphenols in most over-the-counter available nutraceutical preparations, each induced an almost complete clonal suppression. To confirm and extend this observation parallel experiments were set using the HPV-16 transformed Siha cells that yielded almost super-imposable data (Data not shown). Interestingly, in addition to the reported anti-clonal effect, a sharp antiproliferative effect on established tissue cultures could be shown for the compound no. 12) luteolin. A distinct, although milder effect could be also shown for compound no. 9) Kaempferol and no. 16) Apigenin (FIG. 2, panel B)

To quantitate these clonal inhibitory effects limiting dilution experiments were run to determine their 50% clonal inhibitory dose (CID₅₀). The Luteolin (12) and Galangin (10) anti-clonal titration is shown in FIG. 3. The Experimental concentrations are indicated in the left side of plate cover. Luteolin is in columns 1-6 (left side of plate); Galangine is in columns 7-10 on the right side of plate. Control untreated cells are in columns 11-12. An almost complete inhibition can be seen in wells C: 1-6 and a roughly 50% inhibition is clearly evident in wells D:1-6. The percentage of inhibition is best evaluated through live cell image analysis plotted in panel (b) and (c). As it can be seen a 6.25 μM CID₅₀ could be measured for Luteolin while a 25 μM CID₅₀ was shown for Galangin. Higher concentrations of both compounds were completely suppressive.

Based on these evidence it is plausible that a core preparation including the two most powerful anti-clonal compounds Luteolin and Galangin and the two most powerful antiproliferative agents, again Luteolin and Kaempferol, at even or at different molar concentration, might have a powerful anticlonogenic and antiproliferative effect thus representing a new pharmacological formulation to prevent the implantation of HPV transformed cells and their subsequent proliferation and tissue invasion. This is also confirmed by the evident synergistic effect of their triple combination shown in FIG. 12.

In addition to their direct anti-clonal effects the compounds 14 Chrysin, 15 Quercetin and 16 Apigenin once administered in association with each other or with Kaempferol, Galangin and Luteolin, can have a further synergistic effect. Indeed, the Chrysin/Quercetin synergism is shown in FIG. 5, the Kaempferol/Apigenin one is shown in FIG. 4 and the Luteolin/Chrysin and Luteolin/Apigenin synergisms are displayed in FIGS. 7 and 8.

Based on this further piece of evidence, it is plausible that a second set of compounds, made of 14) Chrysin, 15) Quercetin and 16) Apigenin once combined with the core preparation, either isolated or in combination and in yet undefined proportion, might confer a distinct synergistic improvement to the pharmacodynamic activity of the core preparation.

A third set made of the 1-7 molecules, namely the Vanillic, Caffeic, Coumaric, Ferulic, Benzoic, Chlorogenic and Protocatechuic acids, although per se devoid of. any direct anticlonogenic activity are able, as a combination with the core compounds and/or with those of the second set, to induce a certain grade of inhibition: indeed, Vanillic and Caffeic acids have a synergistic effect with Galangine and Apigenin (FIG. 9) and Coumaric, Ferulic, Benzoic, Chlorogenic and Protocatechuic acid synergize with Galangine, Apigenin and Chrysin (FIG. 10-11). As it can be seen in FIG. 12, almost 24 hours are needed to RNP9, RNP10 or RNP12 to induce a roughly 50% of clonal inhibition. Conversely, with the triple combination the clonal activity is readily inhibited with only a 8 hours treatment and its extent clearly exceed 50% with a 16 hours long treatment. Finally, the clonal inhibition achieved by the triple combination after 24 hours was sharper than those of single components. The triple combination thus outperforms its single components in terms of a shorter time of induction and in terms of extent of effect. Similar results were also obtained with HeLa cells. Thus, this third set of compounds holds the promise to realize a convenient in vivo potentiating effect.

As mentioned above, the experiments have been carried out on different HPV cell lines belonging to different HPV types such as HPV 16, HPV 18 and HPV 68. The experiments make it plausible for a person skilled in the art that the advantageous results mentioned above can be obtained for any kind of Alpha HPV infections and related diseases.

Indeed, viral strains are generally distinguished and classified through differences in their natural host range, cell and tissues selective tropism, pathogenicity and cytopathology, structural and chemical-physical properties, antigenic identity and protein and genomic sequence relatedness.

However, Papillomaviruses (PV) have posed an exception to the classical rules of taxonomy since their initial reports (Gissmann and zur Hausen, 1976; Favre et al., 1975; Orth et al., 1978). These differences became increasingly large with growing knowledge and eventually led to the development of a specific and dedicated set of taxonomic criteria which are indeed quite different from the standard ones (de Villiers E M 2013)(Munoz N. et al. 2003)(Castle P E 2009), https://pave.niaid.nih.gov/analyze/11_taxonomy_tool. The core reason for such an exceptional condition lay in the fact that none of the over 200 types of known HPVs (and only a handful of the hundreds of animal PV) can be actually cultivated with ordinary techniques as it is for most of other viral strains and biological entities. Therefore, the structural and functional data needed for a standard classification are simply unavailable and the whole PV classification had to be entirely based on merely genomic data. Thus, the usual categories of variants, types, species, genera and families could not be based on the observation of truly autonomous biological entities but rather on arbitrary numerical criteria of homology. For example, two different isolates of PV are assumed to belong to different species provided they diverge each other by more than 30% of bases in their L1 regions or, for instance, are assigned to two variants of the same type once they diverge by less than 2%. Therefore, HPV types are not referred to clearly different living entities as it would be with Human HerpesVirus type 1 (HSV-1) or type 2 (HSV-2). Rather HPV types are referred to “arbitrarily” distinguishable theoretical entities. To reflect this condition pathologists and clinicians generally refer to HPVs by the vernacular term of “Type” that is used instead of the more correct one of “Species”. However, although entirely based on statistical considerations, this taxonomic system has a clinical relevance in that all the HPV types relevant to human pathology are actually included in the Alphapapillomavirus genus. The seven HPVs types 16; 31; 33; 35; 52; 57 and 58 that belong to the species alpha-9, and the six types 18; 39; 45; 59; 68 and 70 belonging to the species alpha-7, are indeed members of a single pathological class sharing a common anatomical target and inducing a rather homogeneous range of pathological alterations (i.e. CIN-I, CIN-II CIN-III) and are responsible, as a group, of almost the totality of human cervical cancers (Castle P E 2009). Their affinity is further remarked by the fact that a Nona-valent HPV vaccine including 7 of them can confer cross reactive protection to an extended set of related Oncogenic types. (Huh W K et al 2017). Moreover, the two HPV types 6 and 11 and belonging to the related species alpha-10, although responsible of Genital Condilomata (i.e.: benign epithelial proliferation instead of malignant neoplastic lesions), share the same genomic organization and the same target tissue of species alpha-9 and alpha-7 and the spectra of biochemical functions of their early proteins are closely related and partly overlapped. Accordingly, these types, namely type 6; 11; 16; 18; 31; 33, 35; 39; 45; 52; 57; 58; 59; 68 and 70 are currently considered as a rather homogeneous group of parasites and indicated with the term of Ano-genital PVs.

Finally, it has to be remarked that the vast majority of biochemical and molecular biology data on AlphaPV proteins are almost entirely derived from studies based on the expression of subgenomic fragments either from HPV16 or from HPV18 genomes assayed through extensively engineered techniques such as cell transient or stable transfections and lentivirus related expression systems.

Conversely only a very minor part of data is derived from experiments with a few, spontaneously immortalized cell lines. And these few cell lines invariably harbor either the HPV16 genome (SiHa; Ca-Ski; etc) or the HPV18 genome (HeLa; KB, etc). The sole and notable exception to this rule is ME-180 cells, a spontaneously immortalized cell line harbouring the alpha-10 HPV type 68 genome. No other continuous line hosting any other HPV genome is available for cell biology experiments. Thus, at best of current technical tools and scientific knowledge the data here reported are based on the largest available range of experimental cell models and the comments and speculations here proposed are based on the state of art of scientific knowledge on HPV biology. Therefore, for a person skilled in the art, the above-mentioned experimental results would be plausible in connection to human Alpha Papillomavirus genus as a whole.

### REFERENCES

Abel S D A, Baird S K. Honey is cytotoxic towards prostate cancer cells but interacts with the MTT reagent: Considerations for the choice of cell viability assay. Food Chem. 2018 Feb. 15; 241:70-78. doi: 10.1016/j.foodchem.2017.08.083. Epub 2017 Aug. 24.
Abel S D A, Dadhwal S, Gamble A B, Baird S K. Honey reduces the metastatic characteristics of prostate cancer cell lines by promoting a loss of adhesion. PeerJ. 2018 Jul. 3; 6:e5115. doi: 10.7717/peerj.5115. eCollection 2018.
Baker C. C., Phelps W. C., Lindgren V., Braun M. J., Gonda M. A., Howley P. M. Structural and transcriptional analysis of human papillomavirus type 16 sequences in cervical carcinoma cell lines. J. Virol. 61:962-971(1987). PubMed=3029430: DOI=10.1128/JVI.61.4.962-971.1987
Boukamp P, Petrussevska R. T, Breitkreutz D, Hornung J, Markham A, Fusenig N E. Normal keratinization in a spontaneously immortalized aneuploid human keratinocyte cell line. J. Cell Biol. 1988. 106: 761-771.
Cardona F, Andrés-Lacueva C, Tulipani S, Tinahones F J, Queipo-Ortuño M I. Benefits of polyphenols on gut microbiota and implications in human health. J Nutr Biochem. 2013 August; 24(8):1415-22. doi: 10.1016/j.jnutbio.2013.05.001. PMID: 2384945
Castle Philip E. The evolving definition of carcinogenic human papillomavirus. Infectious Agents and Cancer 2009, 4:7 doi:10.1186/1750-9378-4-7.
De Marco F, Perluigi M, Foppoli C, Blarzino C, Cini C, Coccia R, Venuti A. UVB irradiation down-regulates HPV-16 RNA expression: Implications for malignant progression of transformed cells. Virus Res. 2007. 130 (1-2):249-259.
Favre, M., Orth, G., Croissant, O., Yaniv, M., 1975. Human papillomavirus DNA: physical map. Proc. Natl. Acad. Sci. USA 72, 4810-4814.
Friedl F, Kimura I, Osato T, Ito Y. Studies on a new human cell line (SiHa) derived from carcinoma of uterus. I. Its establishment and morphology”. Proc. Soc. Exp. Biol. Med. 1970. 135:543-545(DOI=10.3181/00379727-135-35091a.
Gabutti G, d'Anchera E, De Motoli F, Savio M, Stefanati A. Human Papilloma Virus Vaccination: Focus on the Italian Situation. Vaccines 2021, 9, 1374. https://doi.org/i0.3390/vaccines9121374
zur Hausen. Papillomaviruses in the causation of human cancers—a brief historical account. Virology 384 (2009) 260-26.
Gissmann, L., zur Hausen, H., 1976. Human papillomavirus: physical mapping and genetic heterogeneity. Proc. Natl. Acad. Sci. USA 73, 1310-1313.
Ho H H, Chang C S, Ho W C, Liao S Y, Wu C H, Wang C J. 2010. Anti-metastasis effects of gallic acid on gastric cancer cells involves inhibition of NF-_B activity and downregulation of PI3K/AKT/small GTPase signals. Food and Chemical Toxicology 48:2508_2516 DOI 10.1016/j.fct.2010.06.024.
Huh W K, Joura E A, Giuliano A R, Iversen O E, de Andrade R P, Ault K A et al. Lancet. 2017 Nov. 11; 390(10108):2143-2159. doi: 10.1016/S0140-6736(17)31821-4. Epub 2017 Sep. 5. PMID: 28886907 Clinical Trial.
Kopustinskiene D M, Jakstas V, Savickas A, Bernatoniene J. Flavonoids as Anticancer Agents Nutrients. 2020 Feb. 12; 12(2):457. doi: 10.3390/nu12020457. PMID: 32059369
Kozkowska A, Szostak-Wegierek D. Flavonoids—food sources and health benefits. Rocz Panstw Zaki Hig 2014; 65(2):79-85. PMID: 25272572
Lansky E P, Harrison G, Froom P, Jiang W G. 2005. Pomegranate (Punica granatum) pure chemicals show possible synergistic inhibition of human PC-3 prostate
   cancer cell invasion across Matrigel. Investigational New Drugs 23:121-122 DOI 10.1007/s10637-005-5856-7.
Leri M, Scuto M, Ontario M L, Calabrese V, Calabrese E J, Bucciantini M, Stefani M. Healthy Effects of Plant Polyphenols: Molecular Mechanisms. Int J Mol Sci. 2020 Feb. 13; 21(4):1250. doi: 10.3390/ijms21041250. PMID: 32070025
Lu Y, Jiang F, Jiang H, Wu K, Zheng X, Cai Y, Katakowski M, Chopp M, To S S T. 2010. Gallic acid suppresses cell viability, proliferation, invasion, and angiogenesis in human glioma cells. European Journal of Pharmacology 641(2-3):102-107 DOI 10.1016/j.ejphar.2010.05.043.
Munoz N. et al. 2003 Epidemiologic Classification of Human Papillomavirus Types Associated with Cervical Cancer N Engl J Med 2003; 348:518-27.
Orth, G., Jablonska, S., Favre, M., Croissant, O., Jarzabek-Chorzelska, M., Rzesa, G. 1978. Characterization of two types of human papillomaviruses in lesions of epidermodysplasia verruciformis. Proc. Natl. Acad. Sci. USA 75, 1537-1541.
Scherer W F, Syverton J T, Gey G O. (1953). “Studies on the propagation in vitro of poliomyelitis viruses. IV. Viral multiplication in a stable strain of human malignant epithelial cells (strain HeLa) derived from an epidermoid carcinoma of the cervix”. Journal of Experimental Medicine. 97 (5): 695-710. doi:10.1084/jem.97.5.695. PMC 2136303. PMID 13052828.
Sur S, Panda C K. Molecular aspects of cancer chemo-preventive and therapeutic efficacies of tea and tea polyphenols. Nutrition. 2017 Nov-Dec;43-44:8-15. doi: 10.1016/j.nut.2017.06.006. Epub 2017 Jul. 5.
de Villiers E M. Cross-roads in the classification of papillomaviruses. Virology. 2013 Oct;445(1-2):2-10. doi: 10.1016/j.virol.2013.04.023. Epub 2013 May 16. PMID: 23683837
Yee C., Krishnan-Hewlett I., Baker C. C., Schlegel R., Howley P. M. Presence and expression of human papillomavirus sequences in human cervical carcinoma cell lines. Am. J. Pathol. 119:361-366(1985) PubMed=2990217
Zhou Y, Zheng J, Li Y, Xu D P, Li S, Chen Y M, Li H B. Natural Polyphenols for Prevention and Treatment of Cancer. Nutrients. 2016 Aug. 22; 8(8):515. doi: 10.3390/nu8080515. PMID: 27556486

1. Pharmaceutical composition comprising or consisting of luteolin, galangin and kaempferol, together with one or more excipients and/or adjuvants.

2. Pharmaceutical composition according to claim 1, wherein luteolin is present in an amount ranging from 1 to 20 μM, preferably from 2 to 12 μM, more preferably from 4 to 7 μM, galangin is present in an amount ranging from 10 to 40 μM, preferably from 20 to 40 μM, more preferably from 20 to 30 μM, even more preferably 25 μM, kaempferol is present in an amount ranging from 10 to 40 μM, preferably from 20 to 40 μM, more preferably 22 to 30 μM, even more preferably 12 to 30 μM, for example 25 μM or 15 μM.

3. Pharmaceutical composition according claim 1, wherein said pharmaceutical composition further comprises one, more than one or all the compounds selected from chrysin, quercetin and apigenin.

4. Pharmaceutical composition according to claim 3, wherein chrysin is present in an amount ranging from 10 to 50 μM, preferably from 15 to 30 μM, more preferably 20 μM or 25 μM. quercetin is present in an amount ranging from 40 to 80 μM, preferably from 50 to 60 μM, mor preferably 50 μM. apigenin is present in an amount ranging from 10 to 50 μM, preferably from 10 to 40 μM, more preferably from 15 to 30 μM, more preferably 25 μM.

5. Pharmaceutical composition according to claim 1, wherein said pharmaceutical composition further comprises one, more than one or all compounds selected from vanillic acid, caffeic acid, coumaric acid, ferulic acid, benzoic acid, chlorogenic acid, protocatechuic acid, pinocembrin, rutin and pinobanksin, preferably caffeic acid and/or ferulic acid.

6. Pharmaceutical composition according to claim 5, wherein each of vanillic acid, coumaric acid, benzoic acid, chlorogenic acid, protocatechuic acid, pinocembrin, rutin and pinobanksin compounds is present in an amount <0,100 mM, whereas each of Caffeic acid and Ferulic Acid compounds is present in an amount from 20 to 100 μM.

7. (canceled)

8. A method of treating a viral infection and/or related disease in a patient in need thereof comprising administering to the patient a pharmaceutical composition comprising luteolin, galangin and kaempferol, together with one or more excipients and/or adjuvants.

9. The method of claim 8, wherein the viral infection is HPV infection, such as human Alpha HPV infection, and the related disease is chosen from the group consisting of HPV-related cancer, HPV dysplastic lesions, HPV laryngeal papillomatosis, HPV Genital condylomata, AlphaPapillomavirus sub-clinical anogenital and cervico-vaginal infection and AlphaPapillomavirus unapparent anogenital and cervico-vaginal infection.

10. The method of claim 8, wherein said pharmaceutical composition is administered by a route chosen from the group consisting of topical route, intravenous route, intramuscular route, organ selective extracorporeal perfusion, intracavital perfusion, intra peritoneal perfusion, intracerebral ventricles and subarachnoid continuous perfusion.

11. The method of claim 8, wherein said pharmaceutical composition is delivered by liposomes, nanoparticles; radiofrequencies, electroporation, ionophoresis or shockwaves delivery systems.

12. A method of treating a viral infection and/or related disease in a patient in need thereof comprising administering to the patient a ombination of luteolin, galangin and kaempferol; wherein the luteolin, galangin and kaempferol can be administered separately or sequentially.

13. The method of claim 12, wherein the viral infection is HPV infection, such as human Alpha HPV infection, and the related disease is chosen from the group consisting of HPV-related cancer, HPV dysplastic lesions, HPV laryngeal papillomatosis, HPV Genital condylomata, AlphaPapillomavirus sub-clinical anogenital and cervico-vaginal infection and AlphaPapillomavirus unapparent anogenital and cervico-vaginal infection.
